(19) United States
(12) Patent Application Publication
(10) Pub. No.: US 2018/0333288 A1
(43) Pub. Date: Nov. 22, 2018

(54) Pressure-Distributing Wound Prevention and Treatment Device

(71) Applicant: Avent, Inc., Alpharetta, GA (US)

(72) Inventors: Ryan A. Squires, Tigard, OR (US); John P. Gann, Portland, OR (US); David C. Blessing, Portland, OR (US); Christopher W. Mellick, Wilsonville, OR (US)

(21) Appl. No.: 15/778,018

(22) PCT Filed: Mar. 8, 2016
(86) PCT No.: PCT/US16/21332

§ 371 Date: May 22, 2018

Related U.S. Application Data
(60) Provisional application No. 62/262,971, filed on Dec. 4, 2015.
(60) Provisional application No. 62/262,973, filed on Dec. 4, 2015.

(51) Int. Cl. A61F 5/30; A61F 13/00; A61M 35/00; A61F 13/10; A61F 13/06; A61F 2/78; A61L 15/42; A61L 15/22; A61L 15/18; A61L 15/44; A61L 15/60; A61L 15/46

(52) U.S. Cl. CPC A61F 5/30; A61L 2300/414; A61F 13/00042; A61F 13/00038; A61F 13/00017; A61F 13/00063; A61M 35/00; A61F 13/105; A61F 13/069; A61F 13/068; A61F 13/101; A61F 13/061; A61F 13/104; A61F 2/7812; A61L 15/425; A61L 15/225; A61L 15/18; A61L 15/44; A61L 15/60; A61L 15/46; A61M 2202/0208; A61M 2202/0266; A61M 2202/0275; A61M 2202/0225; A61M 2202/025; A61F 2013/00323; A61F 2002/785; A61F 2013/00272; A61L 2300/404; A61L 2300/402; A61F 13/00025

(57) ABSTRACT

A dressing or device to distribute pressure for the purpose of prevention and treatment of wounds and intact skin is provided. The pressure distributing properties are achieved by a closed cell foam material that is in direct contact with the intact skin or wound and that can also have the ability to manage and absorb moisture. These unique characteristics creates a flexible, and easy to use dressing that can be cut to size and applied to any desired location on a patient.

### RELATED APPLICATIONS

The present application claims priority to U.S. Provisional Application Ser. No. 62/262,973, filed on Dec. 4, 2015, which is incorporated herein in its entirety by reference thereto.

### FIELD OF THE INVENTION

This invention generally relates to wound care, especially the prevention and treatment of pressure-related wounds.

### BACKGROUND OF THE INVENTION

Pressure-related wounds are a significant concern in the healthcare industry. A person's lack of mobility, even for a short period of time, increases the person's risk of developing local ischemia and pressure ulcers. This is of particular concern for people who are bedridden or confined to a wheelchair, where constant pressure against bony protrusions or weak underlying vasculature of the body reduces blood flow to local tissue and removing waste products via the lymphatic system which causes break down in the tissue and skin. These degrading effects are worsened in diabetics who suffer from peripheral tissue ischemia and neuropathy.

It has long been understood that distributing pressure and eliminating pressure points reduces the chance of developing these wounds and increases the chance of complete healing in wounded tissue. A variety of different pressure-distributing devices exist in the field today. In the case of preventing tissue damage from excessive pressure, specialized pressure regulating beds have been developed to create a favorable microclimate with reduced pressure. Further, immobilizing casts can often be applied to patient population at-risk for developing ulcers of the foot, or for people with ulcerations present. Additionally, these abovementioned examples and many others are employed by healthcare professionals in methods for the prevention and treatment of pressure ulcers.

In addition to the foregoing devices, it would be desirable to incorporate pressure distributing qualities into an inexpensive, portable, and flexible form. Previous patents have attempted to address this opportunity, such as in U.S. Pat. No. 4,699,134, which discloses a pressure relieving bandage that includes four distinct layers. However, no absorbent or moisture management properties are described and the construction requires multiple, distinct layers to achieve the intended outcome.

U.S. Patent Application Publication No. 2012/0199134 discloses a heel offloading apparatus designed to relieve pressure around the heel and ankle area. Once again, however, no absorbent or moisture management properties are described.

U.S. Pat. No. 7,182,085 discloses a pressure relieving wound dressing that comprises an absorbent element and a separate non-absorbing pressure distributing element, but no combined (single element, material, or layer) absorbent and pressure relieving device is described. Additionally, the spatial configuration of the absorbent material does not allow constant contact with the wound, resulting in variation in the consistency of treatment.

U.S. Pat. No. 8,075,537 discloses a tissue treatment device arranged in a cell layer, wherein the cells are separated by space or walls, and an occlusive layer. However, the disclosure does not claim any pressure distributing qualities of the device presented.

U.S. Patent Application Publication No. 2014/0305004 discloses a protective footwear insert formed from multiple foam layers and designed to provide cushioning and shock absorption to the foot. The disclosure does not address materials capable of managing moisture, which is key for a successful wound treatment device.

WO/2006/091735 discloses a load relieving dressing with an opening to offload pressure around a wound. The disclosure does not address a primary contacting surface for the wound which can manage moisture and provide a protective barrier to the environment.

As such, a need exists in the field of wound care for wound dressing formed from a single material that is capable of both directly contacting the wound to provide an optimal healing environment and providing pressure relief through distribution of an applied load. The benefits of a single material device over the existing, multi-layered treatment devices would be numerous, including the constancy of treatment, constant contact to the wound, the ability to cut the dressing to size without loss of properties, and the ease of use in the prevention and treatment of pressure ulcers.

### SUMMARY OF THE INVENTION

In accordance with one embodiment of the present invention, a pressure-distributing device is provided. The device includes a biocompatible polymeric matrix, where the matrix is hydrophilic and comprises a polymer and closed cells. The pressure-distributing device is configured to distribute pressure away from an area of intact skin or a wound, where the device is configured to directly contact the area of intact skin or the wound.

In one particular embodiment, the intact skin or the wound is subjected to a resulting pressure level ranging from about 0 pounds per square inch (0 kiloPascals) to about 5 pounds per square inch (34.5 kiloPascals).

In one particular embodiment, the intact skin or the wound can be subjected to a resulting pressure level ranging from about 0 pounds per square inch (0 kiloPascals) to about 5 pounds per square inch (34.5 kiloPascals) despite application of a load having a pressure level ranging from about 1 pound per square inch (6.9 kiloPascals) to about 50 pounds per square inch (345 kiloPascals) to the pressure-distributing device.

In another embodiment, the device can have a thickness ranging from about 0.1 inches (2.54 millimeters) to about 1 inch (25.4 millimeters).

In another particular embodiment, the matrix absorbs moisture from the area of intact skin or the wound.

In still another embodiment, the matrix supplies moisture to the area of intact skin or the wound.

In yet another embodiment, the polymer can be at least partially cross-linked.

In one more embodiment, the polymer can be an absorbent, elastomeric, and biocompatible polymer or copolymer. For instance, the absorbent, elastomeric, and biocompatible polymer or copolymer can include polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof.

Further, the device can also include a polysaccharide. Additionally, the polysaccharide can include dextrin, guar gum, lucerne, fenugreek, xanthan gum, honey locust bean gum, white clover bean gum, carob locust bean gum, carrageenan, sodium alginate, chitin, chitosan, cellulose, or a combination thereof.

In another embodiment, the device can contain a gas, where the gas is contained in the closed cells. In an additional embodiment, a dissolved form of the gas can be contained in or between the closed cells.

In one particular embodiment, the gas can include oxygen, nitrogen, nitric oxide, carbon dioxide, air, helium, or a combination thereof.

In another embodiment, the device of the present invention can further include an active agent. The active agent can include an antimicrobial agent, an antifungal agent, an antifouling agent, an analgesic, a deodorizing agent, nanoparticles, nutrients, growth factors, biologics, or a combination thereof.

In still another embodiment, the device can include a superabsorbent material.

In yet another embodiment, the device can be two-dimensional.

In one more embodiment, the device can be three-dimensional and seamless, where the device conforms to a part of the body having non-planar geometry. Further, the part of the body can be a finger, toe, hand, foot, knee, elbow, heel, amputee stump, or sacral area.

In one particular embodiment, the device can include one or more layers of the biocompatible polymeric matrix.

In an additional embodiment, the device can further include open cells.

A method for preventing or treating pressure sores around an area of intact skin or a wound is also contemplated by the present invention. The method includes directly applying a pressure-distributing device to the intact skin or the wound, where the pressure-distributing device comprises a biocompatible polymeric matrix, where the matrix is hydrophilic and comprises a polymer and closed cells, where the device distributes pressure away from the area of intact skin or the wound.

In one particular embodiment, the intact skin or the wound is subjected to a resulting pressure level ranging from about 0 pounds per square inch (0 kiloPascals) to about 5 pounds per square inch (34.5 kiloPascals).

In one particular embodiment, the intact skin or the wound can be subjected to a resulting pressure level ranging from about 0 pounds per square inch (0 kiloPascals) to about 5 pounds per square inch (34.5 kiloPascals) despite application of a load having a pressure level ranging from about 1 pound per square inch (6.9 kiloPascals) to about 50 pounds per square inch (345 kiloPascals) to the pressure-distributing device.

In another embodiment, the device can have a thickness ranging from about 0.1 inches (2.54 millimeters) to about 1 inch (25.4 millimeters).

In another particular embodiment, the matrix absorbs moisture from the area of intact skin or the wound.

In still another embodiment, the matrix supplies moisture to the area of intact skin or the wound.

In yet another embodiment, the polymer can be at least partially cross-linked.

In one more embodiment, the polymer can be an absorbent, elastomeric, and biocompatible polymer or copolymer. For instance, the absorbent, elastomeric, and biocompatible polymer or copolymer can include polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof.

Further, the device can also include a polysaccharide. Additionally, the polysaccharide can include dextrin, guar gum, lucerne, fenugreek, xanthan gum, honey locust bean gum, white clover bean gum, carob locust bean gum, carrageenan, sodium alginate, chitin, chitosan, cellulose, or a combination thereof.

In another embodiment, the device can contain a gas, where the gas is contained in the closed cells. In an additional embodiment, a dissolved form of the gas can be contained in or between the closed cells.

In one particular embodiment, the gas can include oxygen, nitrogen, nitric oxide, carbon dioxide, air, helium, or a combination thereof.

In another embodiment, the device can further include an active agent. The active agent can include an antimicrobial agent, an antifungal agent, an antifouling agent, an analgesic, a deodorizing agent, nanoparticles, nutrients, growth factors, biologics, or a combination thereof.

In still another embodiment, the device can include a superabsorbent material.

In yet another embodiment, the device can be two-dimensional.

In one more embodiment, the device can be three-dimensional and seamless, where the device conforms to a part of the body having non-planar geometry. Further, the part of the body can be a finger, toe, hand, foot, knee, elbow, heel, amputee stump, or sacral area.

In one particular embodiment, the device can include one or more layers of the biocompatible polymeric matrix.

In an additional embodiment, the device can further include open cells.

A system for preventing or treating pressure sores around an area of intact skin or a wound is also contemplated by the present invention. The system includes a biocompatible polymeric matrix that is hydrophilic and includes a polymer and closed cells. The system distributes pressure away from the area of intact skin or the wound, manages moisture surrounding the area of intact skin or the wound, and delivers a therapeutic agent to tissue beneath the area of intact skin or the wound.

In one embodiment, the intact skin or the wound is subjected to a resulting pressure level ranging from about 0 pounds per square inch (0 kiloPascals) to about 5 pounds per square inch (34.5 kiloPascals).

In an additional embodiment, the intact skin or the wound can be subjected to a resulting pressure level ranging from about 0 pounds per square inch (0 kiloPascals) to about 5 pounds per square inch (34.5 kiloPascals) despite application of a load having a pressure level ranging from about 1 pound per square inch (6.9 kiloPascals) to about 50 pounds per square inch (345 kiloPascals) to the system.

In another embodiment, the matrix can have a thickness ranging from about 0.1 inches (2.54 millimeters) to about 1 inch (25.4 millimeters).

In one more embodiment, the system can absorb moisture from the area of intact skin or the wound.

In still another embodiment, the system can supply moisture to the area of intact skin or the wound.

In yet another embodiment, the polymer can be at least partially cross-linked.

In one more embodiment, the polymer can be an absorbent, elastomeric, and biocompatible polymer or copolymer. For instance, the absorbent, elastomeric, and biocompatible polymer or copolymer can include polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof.

In another embodiment, the matrix further comprises a polysaccharide. The polysaccharide can include dextrin, guar gum, lucerne, fenugreek, xanthan gum, honey locust bean gum, white clover bean gum, carob locust bean gum, carrageenan, sodium alginate, chitin, chitosan, cellulose, or a combination thereof.

In one particular embodiment, the therapeutic agent can be a gas, where the gas is contained in the closed cells.

In still another embodiment, a dissolved form of the gas can be contained in or between the closed cells.

In an additional embodiment, the gas can include oxygen, nitrogen, nitric oxide, carbon dioxide, air, helium, or a combination thereof.

In yet another embodiment, the system can further include an active agent. The active agent can include an antimicrobial agent, an antifungal agent, an antifouling agent, an analgesic, a deodorizing agent, nanoparticles, nutrients, growth factors, biologics, or a combination thereof.

In one more embodiment, the system can further include a superabsorbent material.

In one more embodiment, the matrix can be two-dimensional.

In still another embodiment, the matrix can be three-dimensional and seamless, wherein the matrix conforms to a part of the body having non-planar geometry. The part of the body can be a finger, toe, hand, foot, knee, elbow, heel, amputee stump, or sacral area.

In yet another embodiment, the system can include one or more layers of the biocompatible polymeric matrix.

In one particular embodiment, the matrix can also include open cells.

Other features and aspects of the present invention are discussed in greater detail below.

### BRIEF DESCRIPTION OF THE FIGURES

A full and enabling disclosure of the present invention, including the best thereof to one skilled in the art, is set forth more particularly in the remainder of the specification, including reference to the accompany figures, in which:

FIG. 1 is an illustration showing a cross-section of an absorbent, biocompatible polymeric matrix used to form the pressure-distributing devices contemplated by the present invention;

FIG. 2 shows a cylindrically-shaped three-dimensional pressure-distributing device having openings at opposing ends as contemplated by the present invention;

FIG. 3 shows a cylindrically-shaped three-dimensional pressure-distributing device having an open end and an opposing closed end as contemplated by the present invention;

FIG. 4 shows a three-dimensional pressure-distributing device shaped to receive a protruding part of the body such as an elbow, knee, or heel as contemplated by the present invention;

FIG. 5 shows a planar pressure-distributing device as contemplated by the present invention;

FIG. 6 shows an experimental setup for pressure-mapping of the device of the present invention and the comparative open cell foam wound treatment devices;

FIG. 7 is a graph illustrating the pressure distribution of the device of the present invention as compared to two open cell foam wound treatment devices;

FIG. 8 is another graph illustrating the pressure distribution of the device of the present invention as compared to two open cell foam wound treatment devices;

FIG. 9 is a graph illustrating the pressure distribution of the device of the present invention as compared to two open cell foam wound treatment devices after a 24 hour wet time;

FIG. 10 is another graph illustrating the pressure distribution of the device of the present invention as compared to two open cell foam wound treatment devices after a 24 hour wet time;

FIG. 11 is a graph illustrating the pressure distribution of the present invention compared to a control where no wound treatment device was in place;

FIG. 12 is a graph illustrating the pressure distribution of the device of the present invention as compared to two open cell foam wound treatment devices, where each of the devices tested was a two-layered device having twice the thickness of the devices tested in FIGS. 7-11;

FIG. 13 is a graph illustrating the pressure distribution of a two-layered device of the present invention as compared to two open cell foam wound treatment devices after a 24 hour wet time, where each of the devices tested was a two-layered device having twice the thickness of the devices tested in FIGS. 7-11;

FIG. 14 is a graph illustrating the pressure distribution of a dry single-layered device of the present invention as compared to two dry single-layered open cell foam wound treatment devices;

FIG. 15 is a graph illustrating the reduction in pressure frequency by a dry single-layered device of the present invention as compared to the two dry single-layered open cell foam wound treatment devices;

FIG. 16 is a graph illustrating the pressure distribution of a dry double-layered device of the present invention as compared to two dry double-layered open cell foam wound treatment devices;

FIG. 17 is a graph illustrating the reduction in pressure frequency by a dry double-layered device of the present invention as compared to the two dry double-layered open cell foam wound treatment devices;

FIG. 18 is a graph summarizing the pressure distribution data from FIG. 14 and FIG. 16;

FIG. 19 is a graph illustrating the pressure distribution of a moist single-layered device of the present invention as compared to two moist single-layered open cell foam wound treatment devices;

FIG. 20 is a graph illustrating the reduction in pressure frequency by a moist single-layered device of the present invention as compared to the two moist single-layered open cell foam wound treatment devices;

FIG. 21 is a graph illustrating the pressure distribution of a moist double-layered device of the present invention as compared to two moist double-layered open cell foam wound treatment devices;

FIG. 22 is a graph illustrating the reduction in pressure frequency by a moist double-layered device of the present invention as compared to the two moist double-layered open cell foam wound treatment devices; and

FIG. 23 is a graph summarizing the pressure distribution data from FIG. 19 and FIG. 21.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a pressure-distributing device that can be applied to a wound or an area of intact skin at risk for developing a pressure sore. In one particular embodiment, the pressure distributing device can be applied between an area of intact skin or a wound present on a stump of an amputated limb or appendage and a prosthesis to prevent the development of a pressure sore due to the force applied to the skin or wound by the prosthesis. The pressure-distributing device includes a biocompatible polymeric matrix, wherein the matrix comprises a polymer and closed cells; and a gas contained in the closed cells. By controlling various characteristics of the pressure-distributing device, such as its thickness, its density, the size and number of the closed cells, the Shore hardness, etc., the present inventors have surprisingly found that the device is capable of distributing pressure applied to an area of intact skin or a wound radially away from the area of intact skin or the wound when the pressure-distributing device is applied to the area of intact skin or the wound, such that a pressure level to which the area of intact skin or the wound is subjected is about 0 pounds per square inch (psi). In some embodiments, the device can have a thickness ranging from about 0.1 inches (2.54 millimeters) to about 1 inch (25.4 millimeters), such as from about 0.15 inches (3.81 millimeters) to about 0.99 inches (25.15 millimeters), such as from about 0.25 inches (6.35 millimeters) to about 0.95 inches (24.13 millimeters), such as from about 0.3 inches (7.62 millimeters) to about 0.9 inches (22.86 millimeters), such as from about 0.4 inches (10.16 millimeters) to about 0.8 inches (20.32 millimeters), where the device is capable of distributing pressure applied to an area of intact skin or a wound radially away from the area of intact skin or the wound when the pressure-distributing device is applied to the area of intact skin or the wound such that the area of intact skin or the wound is subjected to a pressure level that ranges from about 0 psi (0 kiloPascals) to about 5 psi (34.5 kiloPascals), such as from about 0.001 psi (0.007 kiloPascals) to about 2.5 psi (17.2 kiloPascals), such as from about 0.001 psi (0.007 kiloPascals) to about 1 psi (6.9 kiloPascals), despite a pressure level ranging from about 1 psi (6.9 kiloPascals) to about 50 psi (345 kiloPascals), such as from about 2.5 psi (17.2 kiloPascals) to about 40 psi (276 kiloPascals), such as from about 5 psi (34.5 kiloPascals) to about 30 psi (207 kiloPascals) being applied to the pressure-distributing device. A method and system for preventing or treating pressure sores around an area of intact skin or a wound are also contemplated by the present invention.

The pressure-distributing device may have a planar or non-planar shape and includes a hydrophilic, biocompatible polymeric matrix comprising a polymer, where the matrix includes closed cells and walls, although it is also to be understood that the cells can be a combination of closed cells and open cells. Unlike traditional closed cell foams, the closed cells contained within the polymeric matrix of the present invention are capable of absorbing moisture from an area of intact skin or a wound when the device is placed in direct contact with the skin or the wound and can also supply moisture to the area of intact skin or the wound. In some embodiments, the matrix is comprised of substantially closed cells. In other embodiments, the matrix contains from about 85% to about 100% closed cells, such as from about 90% to about 99.9% closed cells, such as from about 99% to about 99.5% closed cells.

The pressure-distributing device may also include a gas that can be a therapeutic agent (e.g., a gas such as oxygen), where the gas can be contained in the closed cells and can also be present in dissolved form in moisture in the walls, where, prior to use and in packaged form, moisture can be present in the device in an amount ranging from about 2.5 wt. % to about 15 wt. %, such as from about 3 wt. % to about 10 wt. %, such as from about 5 wt. % to about 9 wt. %. The gaseous and dissolved forms of the therapeutic agent can be produced when, for example, a reactant such as a peroxide is reacted with a catalyst that is a component of the matrix such as a carbonate, resulting in the formation of oxygen gas. Further, the hydrophilic, biocompatible polymeric matrix may be three-dimensional and have a non-planar shape, which allows the wound treatment devices to conform to a part of the body having non-planar geometry, or the biocompatible polymeric matrix may have a planar shape depending on its intended location of use. A method and system for treating a wound or preventing the formation of a pressure wound with a pressure distributing biocompatible polymeric matrix is also described herein.

Although any therapeutic agent (e.g., a gas such as oxygen, nitric oxide, nitrous oxide, carbon dioxide, carbon monoxide, air, etc.) is contemplated as a component of the wound treatment device of the present invention, in one particular embodiment, oxygen can be the therapeutic agent that is utilized. As is known in the art, when oxygen is supplied for the purposes of wound healing, it is known to help with some metabolic processes that rely on oxygen during wound healing. The present invention can generally enhance wound healing by providing oxygen to a wound at greater than atmospheric concentration in conjunction with its pressure distributing properties, which allows the wound treatment device to reduce pressure points on parts of the body that are susceptible to pressure related ulcers. In other words, the present invention encompasses a wound treatment/prevention device capable of distributing pressure away from areas of the body that are more likely to develop pressure ulcers. The device of the present invention can also distribute and absorb a force applied to a wound or injury site (e.g., due to laying on, bumping, etc.) over an increased area, especially if the size of the dressing is larger than the wound or area vulnerable to pressure ulcers, thus providing a protective effect to the wound that can facilitate improved healing of the wound or injury as compared to traditional foam dressings. Insufficient pressure relief can result in tissue damage at the wound or injury site, as new, healed tissue is thin, delicate, and only a few cell layers thick. Applying a force or load to such tissue can undo and delay any healing due to the fragile nature of the new tissue. Meanwhile, the particular components of the closed cell pressure distributing device of the present invention and their arrangement (e.g., the arrangement and size of the cells, the elastomeric nature of the device, and the presence of a gas, etc.) allows for more effective radial distribution of an applied perpendicular load or force such as the type of force exerted by laying on a particular part of the body for too long.

Various embodiments of the present invention will now be described in more detail below.

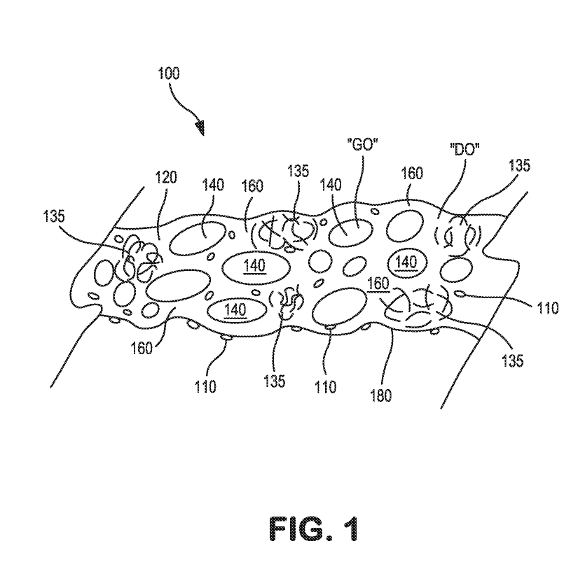

Referring to FIG. 1, there is illustrated in cross-section a portion of an exemplary wound treatment device 100 (not necessarily to scale). The device includes a formed biocompatible polymeric matrix 120. The biocompatible polymeric matrix 120 is used for the distribution of pressure. The matrix may include a water-swellable, cross-linked biocompatible polymer network. Exemplary biocompatible matrices and techniques to form the same are disclosed by U.S. Pat. No. 8,679,523 issued Mar. 25, 2014 and U.S. Pat. No. 7,160,553 issued Jan. 9, 2007 to Gibbins, et al., which are incorporated herein by reference.

The matrix 120 can include closed cells 140 and walls 160. That is, a plurality of gas-permeable, elastic, closed cells 140 can be defined by the cross-linked biocompatible polymer network. However, it is also to be understood that in some embodiments, the matrix 120 can also include a combination of open cells 135 and closed cells 140. In one particular embodiment, the biocompatible matrix may be a biocompatible polymeric matrix that includes a polymer and an oxygen catalyst and the closed cells 140 may be produced from a reaction between the catalyst and a second reactant (e.g., a peroxide). Deliverable oxygen is contained within the elastic closed cells such that when the device is used to treat a wound, oxygen is delivered from the closed cells 140. Generally speaking, gaseous oxygen “GO” is contained in the closed cells 140 and dissolved oxygen “DO” is present in moisture in the walls 160. However, it is to be understood that the therapeutic agent is not limited to oxygen, any therapeutic gases can be utilized in the present invention. For instance, therapeutic agents such as nitric oxide, carbon dioxide, and carbon monoxide in gaseous and dissolved form are also contemplated for use in the wound treatment device of the present invention. Further, although the therapeutic agent can be formed by reacting any suitable catalyst with any suitable reactant as discussed in more detail below, it is also to be understood that the therapeutic agent (e.g., gas) can be introduced to by mixing it into the polymer matrix. In addition, it is to be understood that the matrix is not limited to closed cells and walls, and the matrix can include closed cells or a combination of closed cells and open cells. In addition, also contemplated by the present invention is a three-layered matrix that incorporates a closed-celled foam disposed between a first outer layer and a second outer layer.

Regardless of the particular gas used or the method by which the gas is introduced into the hydrophilic, biocompatible polymeric matrix, the hydrophilic, biocompatible polymeric matrix 120 can be formed from a polymer or copolymer that can be cross-linked into a water-swellable polymer network. By making the cross-linked biocompatible polymer network sufficiently water-swellable, the second reactant (e.g., a peroxide) may be absorbed into the cross-linked biocompatible polymer network.

The cross-linked polymer network should be flexible such that it can define elastic, closed cells that are also gas permeable. A variety of polymers either naturally derived or synthetic may be used to prepare the polymer network. Polymers that are hydrophilic, cross-linkable, and biocompatible are preferred. Exemplary polymers include, but are not limited to, absorbent biocompatible polymers such as polyacrylamide, polyacrylic acid, sodium polyacrylate, polyethylvinyacetate, polyurethane, polyethylene oxide, polyvinyl alcohol, polyvinyl pyrrolidone, or a combination thereof. Other polymers that can be used include polylysine, polyethylene, polybuterate, polyether, silastic, silicone elastomer, rubber, nylon, vinyl, cross-linked dextran, or a combination thereof. The polymer used to form the cross-linked polymer network can be present in an amount ranging from about 0.25 wt. % and 20 wt. %, such as from about 0.5 wt. % to about 15 wt. %, such as from about 1 wt. % to about 10 wt. % based on the total weight of the wound treatment device of the present invention prior to drying. On the other hand, after dehydration, the polymer used to form the cross-linked polymer network can be present in an amount ranging from about 1 wt. % to about 80 wt. %, such as from about 5 wt. % to about 70 wt. %, such as from about 10 wt. % to about 60 wt. % based on the total weight of the wound treatment device of the present invention on a dry-weight basis.

As mentioned above, the polymer network can be suitably lightly crosslinked to render the material substantially water-insoluble. Crosslinking may, for example, be by irradiation or by covalent, ionic, Van der Waals, or hydrogen bonding. Suitable cross-linking agents can include N,N′-methylene-bisacrylamide, bisacrylylycystamine and diallyltartar diamide and can be present in the wound treatment device of the present invention in an amount ranging from about 0.005 wt. % to about 0.5 wt. %, such as from about 0.01 wt. % to about 0.25 wt. %, such as from about 0.025 wt. % to about 0.15 wt. % based on the total weight of the wound treatment device of the present invention prior to drying. After dehydration, the cross-linking agent can be present in an amount ranging from about 0.01 wt. % to about 4 wt. %, such as from about 0.05 wt. % to about 3 wt. %, such as from about 0.1 wt. % to about 2 wt. % based on the total weight of the wound treatment device of the present invention on a dry-weight basis.

The biocompatible matrix may also include a polysaccharide, which can, in some embodiments, be a non-gellable polysaccharide. One example of a suitable polysaccharide is dextrin. Dextrin is a non-cross-linked carbohydrate intermediate between starch and sugars, with the general structure (C₆H₁₀O₅)ₓ, where x can be 6 or 7. In one particular embodiment, type III dextrin can be utilized. In another embodiment, the polysaccharide can be a cellulose or a cellulose derivative. For instance, methyl cellulose (MC), carboxymethyl cellulose (CMC), hydroxyethylcellulose (HEC), hydroxypropylmethyl cellulose (HPMC), hydroxypropyl cellulose (HPC), ethyl hydroxyethylcellulose (EHEC), or a combination thereof can be used in the present invention. In still another embodiment, a galactomannan macromolecule can be used in the wound treatment device. One example of such a macromolecule is guar gum. Other suitable examples of suitable polysaccharides include lucerne, fenugreek, honey locust bean gum, white clover bean gum, carob locust bean gum, xanthan gum, carrageenan, sodium alginate, chitin, chitosan, etc. The polysaccharide can be present in an amount ranging from about 0.005 wt. % to about 25 wt. %, such as from about 0.05 wt. % to about 10 wt. %, such as from about 0.1 wt. % to about 5 wt. %, based on the total weight of the wound treatment device of the present invention prior to drying. On the other hand, after dehydration, the polysaccharide can be present in an amount ranging from about 0.5 wt. % to about 60 wt. %, such as from about 1 wt. % to about 55 wt. %, such as from about 10 wt. % to about 50 wt. % based on the total weight of the wound treatment device of the present invention on a dry-weight basis.

As discussed above, the wound treatment device of the present invention also includes an oxygen catalyst and a second reactant. The catalyst may be sodium carbonate or other alkali and alkali earth compounds may be used provided they are consistent with the product being biocompatible. In addition, more than one catalyst may be used. For instance, one catalyst may be derived from a group consisting of salts of alkali metals and alkali earth metals and the second catalyst may include, but are not limited to, organic and inorganic chemicals such as cupric chloride, ferric chloride, manganese oxide, sodium iodide and their equivalents. Other catalysts, include, but are not limited to enzymes such as lactoperoxidase and catalase. The catalyst can be present in an amount ranging from about 0.005 wt. % to about 5 wt. %, such as from about 0.01 wt. % to about 2.5 wt. %, such as from about 0.05 wt. % to about 1 wt. % based on the total weight of the wound treatment device of the present invention prior to drying. On the other hand, after dehydration, the catalyst can be present in an amount ranging from about 0.01 wt. % to about 10 wt. %, such as from about 0.05 wt. % to about 7.5 wt. %, such as from about 0.1 wt. % to about 6 wt. % based on the total weight of the wound treatment device of the present invention on a dry-weight basis.

Meanwhile, the second reactant used in the wound treatment device of the present invention can be hydrogen peroxide, ammonium peroxide, urea peroxide, sodium peroxide, and other peroxy compounds can be used provided they leave no residue that would be inconsistent with biocompatibility and/or bioabsorption. The present invention contemplates use of components that can generate a gaseous element within the matrix and that are safe and effective for use. For example, an acid catalyst can be incorporated in the matrix followed by perfusion of the matrix with a carbonate to generate carbon dioxide gas within the matrix. The second reactant can be added to the wound treatment device of the present invention in an amount sufficient to create foaming of the wound treatment device to form the dissolved and gaseous oxygen. After dehydration, the second reactant can be present in an amount ranging from 0 wt. % to about 6 wt. %, such as from about 0.001 wt. % to about 3 wt. %, such as from about 0.01 wt. % to about 1 wt. % based on the total weight of the wound treatment device of the present invention on a dry-weight basis.

The biocompatible matrix may further include a plasticizer. The plasticizers may be glycerol/glycerin, water, propylene glycol and/or butanol and combinations thereof may also be used. For example, water can be present in an amount ranging from about 50 wt. % to about 98 wt. %, such as from about 60 wt. % to about 95 wt. %, such as from about 70 wt. % to about 80 wt. % based on the total weight of the wound treatment device of the present invention prior to drying. After dehydration, water can be present in an amount ranging from about 0 wt. % to about 10 wt. %, such as from about 0.1 wt. % to about 5 wt. %, such as from about 0.5 wt. % to about 2.5 wt. % based on the total weight of the wound treatment device of the present invention on a dry-weight basis.

If glycerol is used, the glycerol can be present in an amount ranging from about 0.25 wt. % to about 25 wt. %, such as from about 0.5 wt. % to about 12 wt. %, such as from about 1 wt. % to about 10 wt. % based on the total weight of the wound treatment device of the present invention prior to drying. On the other hand, after dehydration, the glycerol can be present in an amount ranging from about 1 wt. % to about 80 wt. %, such as from about 5 wt. % to about 70 wt. %, such as from about 10 wt. % to about 60 wt. % based on the total weight of the wound treatment device of the present invention on a dry-weight basis.

Although not required, the biocompatible matrix may further include a hydration control agent. The hydration control agent may be an isopropyl alcohol such as isopropanol; however, ethanol, glycerol, butanol, and/or propylene glycol and combinations thereof may also be used. If present, the hydration control agent can be present in an amount ranging from about 0.05 wt. % to about 5% wt. %, such as from about 0.1 wt. % to about 2.5 wt. %, such as from about 0.25 wt. % to about 1 wt. % based on the total weight of the wound treatment device of the present invention prior to drying. On the other hand, after dehydration, the hydration control agent is generally absent from the wound treatment device of the present invention.

Ammonium persulfate and tetramethylethylenediamine (TEMED) may also be included in the wound treatment device of the present invention. The ammonium persulfate can be a component of the wound treatment device of the present invention in an amount ranging from about 0.005 wt. % to about 0.5 wt. %, such as from about 0.01 wt. % to about 0.25% wt. %, such as from about 0.025 wt. % to about 0.1 wt. % based on the total weight of the wound treatment device of the present invention prior to drying. After dehydration, the ammonium persulfate can be present in an amount ranging from about 0.025 wt. % to about 5 wt. %, such as from about 0.05 wt. % to about 3% wt. %, such as from about 0.1 wt. % to about 1 wt. % based on the total weight of the wound treatment device of the present invention on a dry-weight basis. Additionally, the TEMED can be a component of the wound treatment device of the present invention in an amount ranging from about 0.001 wt. % to about 0.5 wt. %, such as about 0.01 wt. % to about 0.25% w/w, such as about 0.025 wt. % to about 0.15 wt. % based on the total weight of the wound treatment device of the present invention prior to drying. On the other hand, after dehydration, the TEMED can be present in an amount ranging from about 0.01 wt. % to about 3 wt. %, such as from about 0.025 wt. % to about 2 wt. %, such as from about 0.05 wt. % to about 1 wt. % based on the total weight of the wound treatment device of the present invention on a dry-weight basis.

Further, one or more active agents can be incorporated into the wound treatment device of the present invention. As shown in FIG. 1, the one or more active agents 110 can be present on at least a skin-contacting portion 180 of the matrix 120. For example, the active agent 110 can be incorporated throughout the matrix 120 as a result of inclusion in the matrix 120 during compounding. Alternatively and/or additionally, the active agent 110 can be applied to the matrix 120 utilizing dipping, coating, deposition, or spraying processes or other similar known techniques.

The active agents incorporated into the present invention are selected on the basis of the use of the device. Active agents and their effects are known by those skilled in the art and methods for including these agents into the matrices of the present invention are taught herein. The present invention contemplates the inclusion of one or more active agents, depending on the intended use. The compositions and devices may include one active agent, or may include multiple active agents.

If the devices are used for topical treatments, such as treatments for compromised tissues, the devices comprise active agents that aid in treatment of compromised tissues. For example, the devices are used for the treatment of wounds, in skin healing or for cosmetic applications. In such devices, the active agents can aid and improve the wound healing process.

Active agents include, but are not limited to, pharmaceuticals, biologics, chemotherapeutic agents, herbicides, growth inhibitors, anti-fungal agents, anti-bacterial agents, anti-viral agents, anti-fouling agents, anti-parasitic agents, mycoplasma treatments, deodorizing agents, growth factors, proteins, nucleic acids, angiogenic factors, anaesthetics, analgesics, mucopolysaccharides, metals, wound healing agents, growth promoters, indicators of change in the environment, enzymes, nutrients, vitamins, minerals, carbohydrates, fats, fatty acids, nucleosides, nucleotides, amino acids, sera, antibodies and fragments thereof, lectins, immune stimulants, immune suppressors, coagulation factors, neurochemicals, cellular receptors, antigens, adjuvants, radioactive materials, nutrients, nanoparticles, or therapeutic agents produced by a catalyst, any other agents that effect cells or cellular processes, or a combination thereof.

Examples of antimicrobial agents that can be used in the present invention include, but are not limited to, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

Growth factor agents that may be incorporated into compositions and devices of the present invention include, but are not limited to, basic fibroblast growth factor (bFGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), interleukin-8 (IL-8); granulocyte-macrophage colony stimulating factor (GM-CSF); the interleukins, and the interferons.

Other agents that may be incorporated into compositions and devices of the present invention are acid mucopolysaccharides including, but are not limited to, heparin, heparin sulfate, heparinoids, dermatitin sulfate, pentosan polysulfate, chondroitin sulfate, hyaluronic acid, cellulose, agarose, chitin, chitosan, dextran, carrageenan, linoleic acid, and allantoin.

Proteins that may be especially useful in the treatment of compromised tissues, such as wounds, include, but are not limited to, collagen, cross-linked collagen, fibronectin, laminin, elastin, and cross-linked elastin or combinations and fragments thereof. Adjuvants, or compositions that boost an immune response, may also be used in conjunction with the wound dressing devices of the present invention.

Other wound healing agents that are contemplated in the present invention include, but are not limited to, metallic oxides and nanoparticles such as zinc oxide and silver ions, which have long been known to provide excellent treatment for wounds. Delivery of such agents, by the methods and compositions of the present invention, provide a new dimension of care for wounds.

It is to be understood that in preferred embodiments of the present invention, the active agents are incorporated into compositions and devices so that the agents are released into the environment and/or retained within the device. In topical treatments, the agents are then delivered via transdermal or transmucosal pathways. The incorporated agents may be released over a period of time, and the rate of release can be controlled by the amount of cross-linking of the polymers of the matrices. In this way, the present invention retains its ability to affect the local environment, kill or inhibit microorganisms, boost the immune response, exert other alterations of physiological function and provide active agents over an extended period of time.

In another embodiment of the present invention, active agents are incorporated directly into micro or macro-cavities of the matrix of the wound dressing devices. The agents may be incorporated by absorption of agents by the matrix, and preferably by incorporation during the polymerization of the matrix. Regardless of the manner in which the active agent is incorporated into the wound treatment device of the present disclosure, the active agent can be present in an amount ranging from about 0.1 wt. % to about 25 wt. %, such as from about 0.25 wt. % to about 20 wt. %, such as from about 0.5 wt. % to about 10 wt. % based on the total weight of the wound treatment device of the present invention on a dry-weight basis.

Administering active agents for the treatment of compromised tissue by using the compositions and methods of the present invention overcomes several of the problems of the prior art. First, the present invention avoids the painful re-application of salves and ointments to compromised tissues. The present invention also allows active agents to be delivered directly to the site to prevent the negative impact of system-wide delivery of the agents. In addition, in contrast to an injection of active agents, the present invention provides methods of administering active agents wherein the agents may be removed immediately from the compromised tissue and the administration terminated.

The pressure-distributing device of the present invention may also include one or more superabsorbent materials to facilitate the ability of the device to absorb moisture, where the moisture is captured between the closed cells and is held inside the matrix even upon application of pressure to a point or points along the device. Suitable superabsorbent materials include hydrophilic polymers which, in crosslinked form, are capable of absorbing large quantities of fluids. In particular, hydrophilic polymers useful in this invention are those typically employed to make superabsorbent polymers (SAPs), such as water-absorbent polymers which contain carboxyl moieties. Among preferred carboxyl containing water-absorbent polymers are those derived from one or more ethylenically unsaturated carboxylic acids, ethylenically unsaturated carboxylic acid anhydrides or salts thereof. Additionally, the polymers can include comonomers known in the art for use in water-absorbent resin particles or for grafting onto the water-absorbent resins, including comonomers such as acrylamide, a vinyl pyrrolidone, a vinyl sulphonic acid or a salt thereof, acrylamidopropane sulfonic acid (AMPS), salts thereof, or phosphonic acid containing monomers, a cellulosic monomer, a modified cellulosic monomer, a polyvinyl alcohol, a starch hydrolyzate, the hydrolyzates of acrylamide copolymers, or crosslinked products of hydrolyzates of acrylamide copolymers. Examples of ethylenically unsaturated carboxylic acid and carboxylic acid anhydride monomers include, but are not limited to, acrylic acids, such as acrylic acid, methacrylic acid, ethacrylic acid, alpha-chloro acrylic acid, alpha-cyano acrylic acid, beta-methyl acrylic acid (crotonic acid), alpha-phenyl acrylic acid, beta-aryloyloxy propionic acid, sorbic acid, alpha-chloro sorbic acid, angelic acid, cinnamic acid, p-chloro cinnamic acid, beta-styd acrylic acid (1-carboxy-4-phenyl butadiene-1,3), itaconic acid, citraconic acid, mesaconic acid, glutaconic acid, maleic acid, fumaric acid and maleic acid anhydride. In some embodiments, the carboxyl containing water-absorbent polymers are derived from acrylic acid, methacrylic acid, or a salt thereof, with partially neutralized products of polyacrylic acids and crosslinked products of partially neutralized polyacrylic acids being especially preferred polymers. Mixtures of monomers can be employed.

The device of the present invention may take many physical forms, depending on the various uses of the device. The device may be left in place for about 1 to 5 days and then removed. Further, the device can absorb up to about 5 times its weight in moisture without altering its ability to distribute pressure radially away from an applied load as discussed above. For instance, the device, in some embodiments, can absorb from about 5 times to about 20 times its weight in moisture. Generally, the biocompatible polymer matrix 120 of FIG. 1 can be molded into any suitable planar two-dimensional or non-planar three-dimensional shape, such that the wound treatment device of the present invention can conform to any protruding part of the user's body or any part of the body to which a large amount of weight is being applied in order to provide protective cushioning and/or pressure relief and/or pressure distribution, which relieves or redistributes pressure at specific parts of the body that are experiencing a large amount of force and either prevents ulcers from occurring or aides in preventing the worsening of existing wounds such as pressure ulcers.

Regardless of its particular physical shape, the overall thickness of the pressure-distributing device can be selectively controlled to range from about 0.1 inches (2.54 millimeters) to about 1 inch (25.4 millimeters), such as from about 0.15 inches (3.81 millimeters) to about 0.99 inches (25.15 millimeters), such as from about 0.25 inches (6.35 millimeters) to about 0.95 inches (24.13 millimeters), such as from about 0.3 inches (7.62 millimeters) to about 0.9 inches (22.86 millimeters) such as from about 0.4 inches (10.16 millimeters) to about 0.8 inches (20.32 millimeters) in order to distribute pressure from an applied load radially away from the area point of pressure application. As such, the device is capable of distributing pressure radially away from an area of intact skin or a wound when the pressure-distributing device is applied to the area of intact skin or the wound such that the area of intact skin or a wound is subjected to a pressure level that is about 0 pounds per square inch (psi). In some embodiments, the area of intact skin or the wound is subjected to a pressure level that ranges from about 0 psi (0 kiloPascals) to about 5 psi (34.5 kiloPascals), such as from about 0.001 psi (0.007 kiloPascals) to about 2.5 psi (17.2 kiloPascals), such as from about 0.001 psi (0.0007 kiloPascals) to about 1 psi (6.9 kiloPascals), despite a pressure level ranging from about 1 psi (6.9 kiloPascals) to about 50 psi (345 kiloPascals), such as from about 2.5 psi (17.2 kiloPascals) to about 40 psi (276 kiloPascals), such as from about 5 psi (34.5 kiloPascals) to about 30 psi (207 kiloPascals) being applied to the pressure-distributing device. The thickness of the device that contributes to such radial pressure distribution can be achieved in a single-layer device, a two-layer device, a three-layer device, a four-layer device, or a device having any other suitable number of layers. Further, the density of the density of the device of the present invention, which also contributes to its pressure distribution capabilities, ranges from about 0.6 pounds per cubic foot (9.6 kilograms per cubic meter) to about 2.3 pounds per cubic foot (36.8 kilograms per cubic meter), such as from about 0.7 pounds per cubic foot (11.2 kilograms per cubic meter) to about 2.2 pounds per cubic foot (35.2 kilograms per cubic meter), such as from about 0.8 pounds per cubic foot (12.8 kilograms per cubic meter) to about 2.1 pounds per cubic foot (33.6 kilograms per cubic meter). In addition, the density of the closed cells (and open cells if present) present in the matrix, which also contributes to the device of the present invention's pressure distribution capabilities, ranges from about 4,000 cells per cubic inch (244 cells per cubic centimeter) to about 17,000 cells per cubic inch (1037 cells per cubic centimeter), such as from about 5,000 cells per cubic inch 305 cells per cubic centimeter) to about 15,000 cells per cubic inch (915 cells per cubic centimeter), such as from about 6,000 cells per cubic inch (366 cells per cubic centimeter) to about 12,000 cells per cubic inch (732 cells per cubic inch).

Moreover, in addition to being capable of distributing pressure radially away from a load applied to an area of intact skin or a wound, the device of the present invention having the characteristics discussed above is capable of managing the level or amount of moisture at the surface of the intact skin or wound to prevent tissue maceration. In other words, the device is capable of absorbing enough moisture from the skin or the wound to prevent maceration without excess drying of the skin or the wound as well as supplying or donating moisture to the skin or wound as needed. In this manner, the device is capable of maintaining the appropriate moisture conditions for the prevention and/or treatment of pressure wounds or sores.

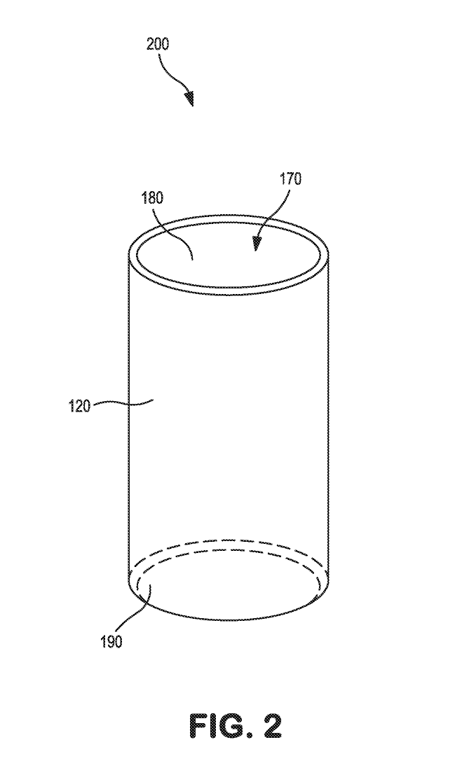

Several embodiments of the pressure-distributing device including the biocompatible polymeric matrix 120 of FIG. 1 are shown in FIGS. 2-5, although it is to be understood that the wound treatment device can be seamlessly molded into any suitable shape to match the protruding or curved part of the body on which it is to be placed. For example, in FIG. 2, the wound treatment device 200 is in the shape of a cylinder, sleeve, or tube. The wound treatment device 200 has a first open end 170 and an opposing second open end 190, such that a protruding part of the body (e.g., hand, arm, finger, leg, foot, toe, etc.) can be inserted through both openings, where the interior of the wound treatment device 200 has a skin contacting surface 180 that can conform around the protruding part of the body without any gaps so that the wound treatment device 200 can provide a therapeutic agent (e.g., a gas such as oxygen) and, if desired, one or more active agents, to a wound.

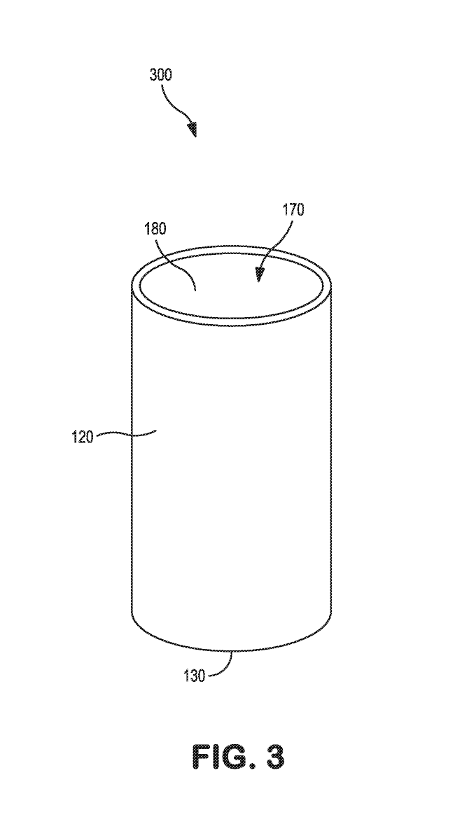

Meanwhile, FIG. 3 shows a wound treatment device 300 that is also in the shape of a cylinder, sleeve, or tube. However, the wound treatment device 300 only has a first open end 170, and an opposing second end 130 that is closed. Such a device 300 can be used for treating a wound on protruding part of the body (e.g., hand, arm, finger, leg, foot, toe, etc.) by inserting the protruding part of the body through the first opening 170, where the interior of the wound treatment device 300 has a skin contacting surface 180 that can conform around the protruding part of the body without any gaps so that the wound treatment device 200 can provide oxygen and, if desired, one or more active agents, to a wound. Further, the closed end 130 of the wound treatment device 300 can provide added protection to an exposed end of the protruding body part. For example, if the user has an amputated arm, the closed end 130 can protect the end of the arm from re-injury, which might occur from bumping or scraping. Such three-dimensional seamless construction is particularly well suited for use on the stump of an amputated limb. By eliminating seams, the device is able to avoid pressure concentration can build up at a seam present across the interface of a stump and prosthesis.

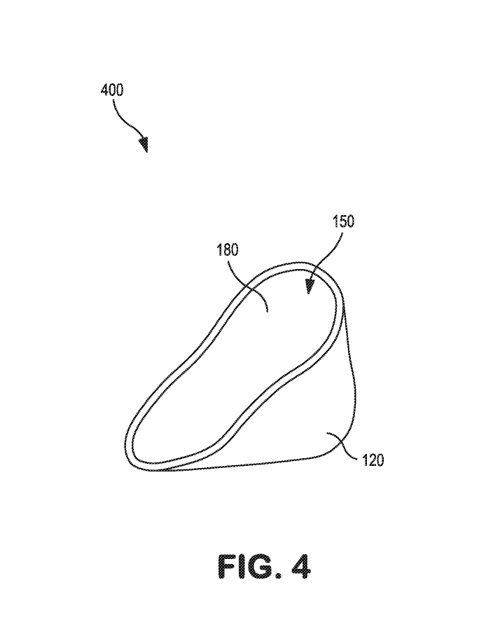

Further, FIG. 4 shows another embodiment of a wound treatment device 400 that has a cone shape. A protruding body part having a cone shape such as an elbow or knee can be inserted into an opening 150 such that a skin-contacting surface 180 of the wound treatment device 400 fits around the elbow or knee. The wound treatment device 400 can also be placed on a heel.

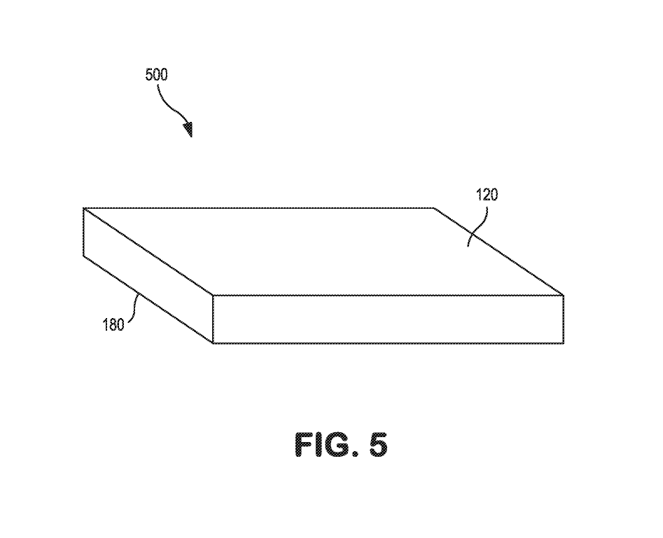

In addition, FIG. 5 shows another embodiment of a wound treatment device 500 contemplated by the present invention. The device 500 is generally planar and made from one layer of material that includes the biocompatible polymeric matrix 120, where a skin contacting surface 180 can be placed on a generally flat or slightly curved area of skin. Further, although not shown, it is also to be understood that the device 500 can include two or more layers of material that include the biocompatible polymeric matrix 120.

The present invention further encompasses a process for making a wound treatment device described above. In one embodiment, the process generally involves at least the steps of: providing a gelling mixture of at least one cross-linkable biocompatible polymer and an oxygen catalyst (e.g., a first reactant); pouring the gelling mixture into a suitable mold to create the desired planar two-dimensional or non-planar three-dimensional shape, cross-linking the biocompatible polymer of the gelling mixture to form a water swellable, cross-linked biocompatible polymer network; drying the gelling mixture to form a molded gel; adding a second reactant to the molded gel; and generating a plurality of closed cells (i.e., closed cells and walls) or a combination of closed cells and open cells containing oxygen in the molded gel by reacting the catalyst and the second reactant.

Generally speaking, the process for making an exemplary wound treatment device of the present invention generates a biocompatible matrix for delivering oxygen. A feature of the biocompatible matrix is the formation of the foam or array of bubbles that entrap the gas. The foam or bubbles are formed by the permeation of the second reactant added to the formed matrix that includes a reactant. When the two reactants interact, a reaction occurs that liberates oxygen gas which becomes entrapped within the matrix. For example, a matrix has a carbonate catalyst (a reactant) incorporated within it. The formed matrix is then placed in the presence of the second reactant (e.g., a peroxide compound such as, for example, hydrogen peroxide). A catalytic decomposition of hydrogen peroxide occurs resulting in the liberation of oxygen gas which becomes entrapped as bubbles formed in situ. The hydrogen peroxide reactant is not part of the compounding of the matrix, but it is in the treatment after the formation of the matrix stock.

According to the process, a gelling mixture of at least one cross-linkable biocompatible polymer is prepared. This may be accomplished by creating a solution of a cross-linkable biocompatible polymer or a solution composed of a mixture of cross-linkable biocompatible polymers. The solution is desirably an aqueous solution or a solution where water component is the major component. According to the process, any of the active agents discussed above may be added to the gelling mixture.

A catalyst for generating the gaseous oxygen (e.g., a first reactant) may be introduced into the gelling mixture. For example, the catalyst may be sodium carbonate, although any of the catalysts discussed above may be utilized. For instance, other catalysts such as other alkali and alkali earth compounds may be used provided they are consistent with the product being biocompatible. In addition, more than one catalyst may be used. For example, one catalyst may be derived from a group consisting of salts of alkali metals and alkali earth metals and the second catalyst may include, but are not limited to, organic and inorganic chemicals such as cupric chloride, ferric chloride, manganese oxide, sodium iodide and their equivalents. Other catalysts, include, but are not limited to enzymes such as lactoperoxidase and catalase. Desirably, the catalyst is a material that interacts with the second reactant.

The polysaccharide, plasticizer and/or a hydration control agent may then be added to the gelling mixture.

The cross-linkable biocompatible polymer of the gelling mixture is then cross-linked to form a water swellable, cross-linked biocompatible polymer network. This may be accomplished by activating a cross-linking agent already present in the gelling mixture, adding or applying a cross-linking agent to the gelling mixture, dehydrating or removing solvent from the gelling mixture, and/or otherwise creating conditions in the gelling mixture that causes cross-linking (e.g., pH, heat, various forms of radiation including electromagnetic radiation and x-rays, ultrasonic energy, microwave energy and the like).

For example, the gelling mixture may be cross-linked by activating a cross-linking agent and dehydrating the gelling mixture in the mold, resulting in a molded gel that can be readily handled. As another example, the gelling mixture may be cross-linked by placing the mold in a conventional oven at an elevated temperature (e.g., 45 to 65° C.) and dehydrating for 2 to 20 hours until the gelling mixture reaches a consistency similar to a leathery gum. Fruit leather or fruit roll-up has a gravimetric or weight-based moisture content of about 10 to about 20 percent. Desirably, the resulting molded gel is flexible and elastic, and may be a semi-solid scaffold that is permeable to substances such as aqueous fluids, silver salts, and dissolved gaseous agents including oxygen. Though not wishing to be bound by any particular theory, it is thought that the substances permeate the matrix through movement via intermolecular spaces among the cross-linked polymer.

A second reactant is then added to the molded gel. This may be accomplished by pouring a solution of the second reactant over the molded gel, or by spraying, brushing, coating or applying the second reactant. In one particular embodiment, the second reactant is hydrogen peroxide. The hydrogen peroxide may be used at a concentration ranging from about 1 wt. % to about 30 wt. % or more, such as from about 3 wt. % to about 20 wt. %. Other peroxides may be substituted, including, but not limited to, urea peroxide, sodium peroxide or other peroxy compounds of alkali metal or alkali earth metals. The second reactant preferably is present in aqueous solution or a solution wherein water is major component.

The second reactant is absorbed into the molded gel and permeates the swellable, (e.g., water-swellable) cross-linked biocompatible polymer matrix. The degree of swelling may be increased by adding a polysaccharide such as dextrin, guar gum, xanthan gum, locust bean gum, carrageenan, sodium alginate, methyl cellulose, hydroxypropyl cellulose, carboxymethyl cellulose, etc. as well as by adding a hydration control agent such as glycerol. By making the cross-linked biocompatible polymer network sufficiently water-swellable, the second reactant (e.g., hydrogen peroxide) may be adequately absorbed into the cross-linked biocompatible polymer network.

According to the process of the present invention, when the second reactant is absorbed and permeates into the cross-linked biocompatible polymer matrix, a gas is generated when the catalyst (i.e., the first reactant) reacts with the second reactant. The resulting gas is desirably oxygen gas. The matrix of the present invention forms a foam or array of bubbles that entrap the gas. That is, a plurality of closed cells containing oxygen is generated in the molded gel by reaction between the catalyst (i.e., the first reactant) and the second reactant.

For example, a cross-linked biocompatible polymer matrix may have a carbonate catalyst (i.e., a first reactant) incorporated within it. The cross-linked biocompatible polymer matrix is then placed in the presence of the second reactant, hydrogen peroxide. A catalytic decomposition of hydrogen peroxide occurs resulting in the liberation of oxygen gas which becomes entrapped as bubbles formed in situ. The hydrogen peroxide reactant is not part of the compounding of the matrix, but it is added after the formation of the matrix stock.

In finished form, a planar two-dimensional or non-planar three-dimensional foam is formed that can have any shaped determined by the mold in which the solution is placed. The matrix formed by the process described above can absorb a vertical load or force applied to a wound site, where the absorption of the load force is redistributed radially, which, in turn, facilitates healing of the wound, protection of the wound site, distribution of pressure at the wound site, prevention of reinjury at the wound site, or a combination thereof.

It is contemplated that the process of the present invention may further include the step of heating or adding energy to the molded gel and second reactant to speed up the reaction.

The present invention may be better understood with reference to the following examples. All chemicals used in the examples described below were reagent grade or a higher grade unless specified otherwise.

### Example 1: Matrix Formation

A polymer matrix containing oxygen gas was produced as an exemplary wound treatment device to deliver oxygen. The device was a closed cell foam cylinder, as shown in FIG. 2, with oxygen gas contained in the cells of the foam. The closed cell foam composition/solution for use in forming the wound treatment devices was generally made in accordance with the process described in the Detailed Description above as well as U.S. Pat. No. 7,160,553 issued Jan. 9, 2007 and U.S. Pat. No. 8,679,523 issued Mar. 25, 2014 to Gibbins, et al.

### Gel Preparation

The components used to form the three dimensional, seamless biocompatible polymeric matrices of Example 1, as well as their concentrations, are listed in Table 1 below.

TABLE 1
Oxygen Containing Foam Formulation

| | | | |
|---|---|---|---|
| | | | Estimated |
| | Pre-Drying | Post-Drying | Concentration |
| | Concentration | Concentration | Ranges |
| Component | (Wt. % of Batch) | (Wt. % of Batch) | (Wt. %) |

Gel Matrix Solution

| | | | |
|---|---|---|---|
| H₂O | 84.98 | ~0.0 | 0-10 |
| Glycerin | 5.72 | 38.07 | 10-60 |
| Acrylamide | 5.64 | 37.58 | 10-60 |
| Bis- | 0.07 | 0.46 | 0.01-2 |
| acrylamide | | | |
| Dextrin | 3.28 | 21.82 | 10-50 |
| Na₂CO₃ | 0.17 | 1.16 | 0.1-6 |
| TEMED | 0.08 | 0.53 | 0.05-0.6 |
| Ammonium | 0.068 | 0.38 | 0.05-1.0 |
| Persulfate | | | |

Dip Step

| | | | |
|---|---|---|---|
| H₂O₂ | NA | 0-6 | 0-6 |
| O₂ | NA | 1.4-6.0 | 0.5-20 |

After forming the gel matrix solution using the components listed above, plastic beakers of varying volumes were used as the molds to form the three-dimensional wound treatment devices of Example 1, where the devices contained a biocompatible polymeric matrix formed from a gel matrix solution. The plastic beakers had a smaller beaker placed inside after being filled with the gel matrix solution. This created a mold for forming a seamless, cylindrical gelled tube in each of the beaker assemblies. Eight molds were assembled for this experiment with various volumes. Table 2 lists the volumes of the inner and outer beakers for each of the nine molds used to form the gelled matrices of Samples 1-8.

The purpose of forming these shapes is to cast gelled matrices in shapes besides flat sheet dressings.

TABLE 2
Type of Molds Used for Each Gel

| Sample ID | Outer beaker | Inner beaker |
|---|---|---|
| 1 | 150 mL | 100 mL |
| 2 | 150 mL | 100 mL |
| 3 | 150 mL | 100 mL |
| 4 | 150 mL | 100 mL |
| 5 | 150 mL | 100 mL |
| 6 | 150 mL | 100 mL |
| 7 | 400 mL | 150 mL |
| 8 | 400 mL | 150 mL |

### Drying the Gelled Matrices

Samples 1 and 2

The gelled matrices of Samples 1 and 2 were removed from the outer plastic beakers after 3.5 hours (210 minutes). Sample 1 was attempted to be removed from the inner beaker and tore in the process. Sample 2 was successfully removed without tearing. Both Samples 1 and 2 were placed back onto the inner beakers so that the beakers could provide support for the gelled matrices during the drying step.

The gelled matrices were dried in an oven for 16 hours at 50° C. After removal from the oven, the gelled matrix of Sample 1 was attempted to be removed. The material tore further during this step. The gelled matrix of Sample 2 was left on the inner beaker for the foaming step.

Samples 3 to 8

The gelled matrices of Samples 3 to 8 were removed from the other plastic beakers after about 100 hours. The gelled matrices of Samples 3, 5, and 6 tore during removal from the inner beakers. All were placed back onto the inner beakers for support during drying. The gelled matrices were dried in an oven for 16 hours at 50° C. After removal from the oven, the gelled matrix of Sample 8 was removed from the inner beaker with difficulty but without tearing. The remaining gelled matrices were left on the inner beakers for the foaming step.

### Foaming

Samples 1 and 2

Approximately 50 mL of a 20% peroxide foaming solution was prepared. The peroxide foaming solution was poured into a 150 mL plastic beaker. The gelled matrices of Samples 1 and 2 were submerged into the peroxide foaming solution while on the 100 mL plastic beakers. Table 3 lists the foaming parameters that were used for Samples 1 and 2. The permeation and foaming steps were performed with the material being supported on the 100 mL beakers inverted. Samples 1 and 2 were prepared to investigate the feasibility of creating three-dimensional shapes.

TABLE 3
Foaming Parameters for Samples 1 and 2

| | | | | Foaming |
|---|---|---|---|---|
| | Peroxide Dip | Permeation | Foaming | Time |
| Sample ID | Time (minutes) | Time (minutes) | Temp ° C. | (minutes) |
| 1 | 3 | 10 | 55° C. | 135 |
| 2 | 3 | 10 | 55° C. | 135 |

The foam of Sample 2 was still adhered to the sides of the plastic beaker in places. The foam was in the shape of a cup, similar to FIG. 3 discussed above. The peroxide solution had to diffuse from the outer surface throughout the entire material, unlike in foaming the sheet dressings where it is exposed to the peroxide solution from both sides.

The foam of Sample 1 was not adhered to the beaker after foaming but was still being supported by the beaker. The resulting foam was in the shape of a half cup, which was caused by the tearing of the material during previous processing steps.

Samples 3 to 8

Approximately 250 mL of 20% peroxide foaming solution was prepared. The foaming solution was poured into either 250 mL or 150 mL plastic beakers depending on the sample to be dipped. The gelled matrices of Samples 3 through 9 were submerged into the peroxide foaming solution while on the plastic inner supporting beakers, except that the gelled matrix of Sample 8 was not on the inner beaker for the peroxide dip step. Table 4 lists the foaming parameters that were used for the gelled matrices of Samples 3 through 8. The permeation and foaming steps were performed with the material being supported on the beakers inverted.

TABLE 4
Foaming Parameters for Samples 3 to 8

| Sample | Peroxide dip | Permeation | Foaming | Foaming time |
|---|---|---|---|---|
| ID | time (minutes) | time (minutes) | Temp (° C.) | (minutes) |
| 3 | 4 | 10 | 55° C. | 135 |
| 4 | 4 | 10 | 55° C. | 135 |
| 5 | 4 | 10 | 55° C. | 135 |
| 6 | 4 | 10 | 55° C. | 135 |
| 7 | 10 | 15 | 55° C. | 135 |
| 8 | 10 | 15 | 55° C. | 135 |

After removal from the foaming oven, the resulting foam material was removed from the supporting beakers as needed. The foam of Samples 3, 4, 5, and 6 were generally cup-shaped and similar to FIG. 3 discussed above.

The foam of Samples 7 and 8 was formed into thick foam tubes similar to FIG. 2 discussed above. The foam tubes were large enough to fit over the hand and onto the arm similar to an arm band. The thickness of the walls was not uniform around the circumference of the tube. This was due to the inner beaker not being centered inside of the outer beaker during the polymerization stage. The thicker wall sections were tacky; this can be an indicator that the foaming was not complete in that section of the foam.

### Testing

Samples were taken from the foam of Samples 3, 6, and 8 for oxygen content and absorbency testing. The samples were taken from sections of the foam that best represented the material. The oxygen test required two samples and thickness of the samples varied due to the varying wall thickness.

Absorbency

The absorbency of the foam samples was measured according to the method described below. To test the absorbency of the foam, a 2 centimeter (cm) by 4 cm rectangular sample was cut out of the foam shapes. The “Initial Weight” of the sample was recorded and placed into a 125 mL plastic cup with lid. The plastic cups were filled with USP grade water, capped and inverted. After 24 hours±1 hour the cups were placed upright and opened. The foam sample was placed onto a Kimwipe (Ref. #34721) for 30 seconds. After 30 seconds the sample was flipped and placed onto a dry section of the Kimwipe. After 30 seconds on the opposite side the “Weight After 24 hrs” of the sample was recorded. The increase factor (absorbency) was calculated by dividing the “Weight After 24 hrs” by the “Initial Weight.” The goal was for the foam samples to have a minimum increase factor of 5, which would indicate that the foam samples were able to absorb exudate from a wound in a manner comparable to a two-dimensional sheet wound dressing as described in U.S. Pat. No. 7,160,553 issued Jan. 9, 2007 and U.S. Pat. No. 8,679,523 issued Mar. 25, 2014 to Gibbins, et al.

The absorbency results for the foam samples taken from the dextrin molds for Samples 3, 6, and 8 are listed in Table 5. The foam shapes were tested in the pre-sterilized condition.

TABLE 5
Absorbency Date

| Sample | Initial | Weight After | Increase |
|---|---|---|---|
| ID | Weight (g) | 24 hrs (g) | Factor |
| 3 | 0.2014 | 7.2627 | 36.10 |
| 6 | 0.2910 | 8.8019 | 30.30 |
| 8 | 0.4258 | 8.6185 | 20.24 |

The three samples all met the Minimum requirement for absorbency of 5. While an increase factor of 5 is the minimum absorbency, higher absorbency values are desired. The foam of Sample 8 had variable wall thickness with tacky sections. The foaming in those tacky sections may not have been completed and it could impact the absorbency results.

Oxygen Content

The oxygen content of the foam samples was measured according to the method described below. To test the oxygen content of the foam two 1.5 cm×4 cm rectangular samples were cut out of the foam shapes. The weight of the foam samples was recorded and placed into 300 mL BOD (Biochemical Oxygen Demand) bottles. The bottles were filled with USP grade water that had been aerated and then capped. After 24 hours±1 hour the oxygen content of the water was measured using an Ocean Optics Neofox Phase Fluorometer and FOXY oxygen probe. The instrument provided an extracted oxygen value in ppm that will be referred to as “Neofox O₂ ppm” for each test sample. This value was used to calculate the O₂ ppm of the foam sample for each sample. The goal was for the foam samples to have a minimum O₂ parts per million (ppm) concentration of 14,354 ppm, which would indicate that the foam samples were able to release dissolved oxygen in a manner comparable to a two-dimensional sheet wound dressing as described in U.S. Pat. No. 7,160,553 issued Jan. 9, 2007 and U.S. Pat. No. 8,679,523 issued Mar. 25, 2014 to Gibbins, et al.

The oxygen results for the foam samples for Samples 3, 6, and 8 are listed in Table 6. The foam shapes were tested in the pre-sterilized condition. In Table 6, “Neofox O₂ ppm” is the measurement from the Neofox instrument while “O₂ ppm” is the oxygen content of the foam sample incorporating the sample weight.

TABLE 6
Oxygen Content Data

| | Weight | Neofox | | Average | Extracted |
|---|---|---|---|---|---|
| Sample ID | (g) | O₂ ppm | O₂ ppm | O₂ ppm | O₂ (μg) |
| 3A | 0.2062 | 22.54 | 22,921 | 24,898 | 4028 |
| 3B | 0.1146 | 17.85 | 26,876 | | 2621 |
| 6A | 0.1688 | 18.64 | 19,889 | 15,415 | 2858 |
| 6B | 0.2501 | 16.87 | 10,940 | | 2327 |
| 8A | 0.2936 | 25.31 | 19,409 | 17,816 | 4859 |
| 8B | 0.4190 | 28.44 | 16,222 | | 5798 |

Based upon the Average O₂ ppm values all samples met the oxygen requirement for oxygen-containing foam dressings. While the requirement for oxygen is 14,354 ppm, a higher oxygen content in a dressing is highly desirable. The foam of Sample 6B did not meet the oxygen requirement for an individual sample. The calculation for O₂ ppm takes into account the weight of the sample being tested. The foam shapes prepared for this experiment were thicker than the standard control two-dimensional wound dressings in sheet form, meaning the weight of the samples was considerably more than the standard samples. Thus, when weight is factored into the calculation the oxygen values are low. The last column in Table 6 shows the amount of extracted O₂ during the test. The Extracted O₂ (μg) for a typical two-dimensional sheet wound dressing as described in U.S. Pat. No. 7,160,553 issued Jan. 9, 2007 and U.S. Pat. No. 8,679,523 issued Mar. 25, 2014 to Gibbins, et al. was 1800-2500 μg. Thus, when comparing the Extracted O₂ (μg), the foams of Samples 3 and 6 were comparable to the two-dimensional sheet wound dressing, while the foam of Sample 8, which was the foam tube, had extracted oxygen values that were double the range of the two-dimensional sheet wound dressing.

The results above show that wound treatment devices can be foamed into desirable seamless, three-dimensional shapes in addition to a planar sheet. The foam dressings were prepared into simple shapes of cups and tubes. These shapes can be used to cover a variety of portions of the body. The absorbency of the foam shapes exceeded the minimum requirements. The oxygen content of the foam shapes met the minimum requirement when calculated as parts per million (ppm). Further, analyzing the oxygen content as extracted oxygen in micrograms (μg), shows that the three-dimensional wound treatment devices of the present invention are capable of supplying sufficient quantities of oxygen.

In the present example, although two different foam shapes were created out of the beaker molds, where one of the shapes was a tube shape open on each end and the other shape was a cup shape that was open on one end only creating the bottom of the cup, any other suitable shape for covering a wound on a protruding part of the body are also contemplated. The tube shape could be used to slide over the hand and up the arm or over the foot and ankle and up the leg. Another approach would be to cut the foam along the axial line, wrap around the appendage and cover with a secondary dressing. The cup shape can cover the end of an appendage in the case of an amputation. Meanwhile, smaller foam cups can be made for use on the fingers and thumbs. In another embodiment, the bottom of one of the foam cups could be cut off to create a foam tube.

### Example 2: Pressure Distribution Testing

### Background

The following experiments were carried out on a pressure-distributing device formed in a manner similar to the wound treatment device described above and in U.S. Pat. No. 7,160,553 issued Jan. 9, 2007 and U.S. Pat. No. 8,679,523 issued Mar. 25, 2014 to Gibbins, et al, except that the dressing was in the form of a planar, two-dimensional sheet dressing having a thickness of 0.26 inches (6.60 millimeters), along with two commercially available open cell foam wound dressings to compare the pressure distributing ability of the device of the present invention with two commercially available wound dressings using a semi-quantitative pressure mapping system, where pressure is defined as the perpendicular force applied to a surface per unit area of the surface over which the force is distributed. Pressure on the surface of a human body causes shear stress between the soft tissues and can affect blood flow and metabolism of these tissues. Incorporating pressure absorbing characteristics into a primary wound dressing is desired to protect and support healing of pressure sensitive wounds and provide product differentiation in the advanced wound dressing market.

The three foam dressings tested were an Allevyn® Non-Adhesive (Smith & Nephew) foam dressing, a Mepilex® foam heel dressing (Molnlycke), and the foam dressing of the present invention. As shown below, these dressings differed in composition, which affected their inherent ability to absorb pressure. The Allevyn® foam dressing is comprised of three distinct layers including a low moisture vapor transmission rate (MVTR) top layer, an absorbent hydrocellular polyurethane foam middle, and a non-stick wound contacting layer having a total thickness of 5.59 millimeters (0.22 inches) and a mass of 6.62 grams. The Mepilex® foam heel dressing is an open-cell polyurethane foam with Safetac® Adhesive on one side having a total thickness of 5.59 millimeters (0.22 inches) and a mass of 6.27 grams. Since the Mepilex® foam heel dressing was shaped in a large FIG. 8 or hourglass-like pattern, it was cut in half for these tests. As described above with respect to Example 1, the foam dressing of the present invention is a closed cell absorbent polyacrylamide foam containing oxygen. The foam dressing of the present invention used for testing had a total thickness of 6.60 millimeters (0.26 inches) and a mass of 1.67 grams. Simulated use consisted of exposure to 0.9% saline over a period of time (e.g., 24 hours) to represent fluid management in clinic.

### Experimental Setup

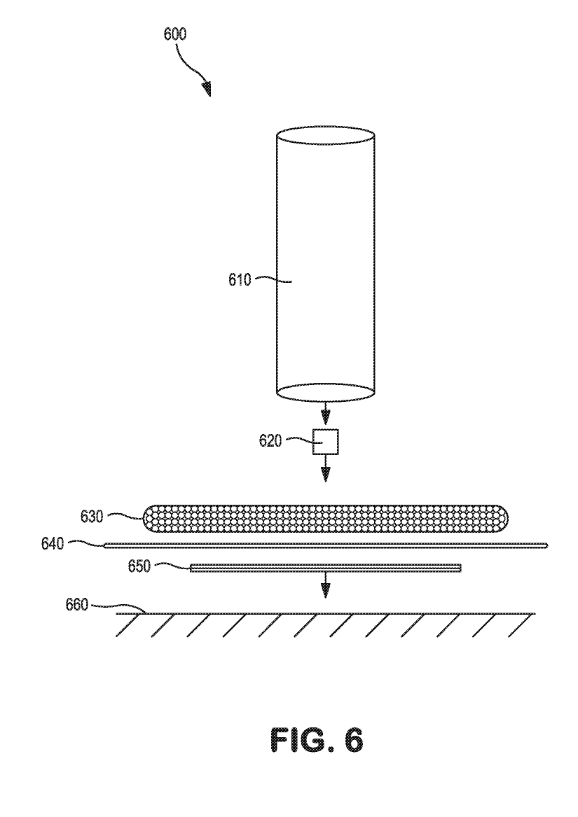

As shown in FIG. 6, Extreme Low FujiFilm Prescale® 7.2-28 psi (Sensor Products Inc., Madison, N.J.) 650 was used to create a visual “color map” of applied pressure through the sample dressings and was used to assess the pressure between the dressings and a concrete floor. The specified pressure range of this film is 7.2 pounds per square inch (psi) (49.6 kiloPascals) to 28 psi (193 kiloPascals). The film 650 is designed to be placed between two contacting surfaces and functions using a single-use donor sheet and a single use receiver sheet, the latter of which changes color in the presence of applied pressure. The magnitude of color change can be correlated to approximate pressure using a color correlation chart provided by the manufacturer and by determining the number of color pixels present on the film after pressure is applied to the film through the sample dressings, where a larger number or frequency of pixels corresponds to an increase in the pressure experienced by the film, which is analogous, for testing purposes, to a wound or intact skin.

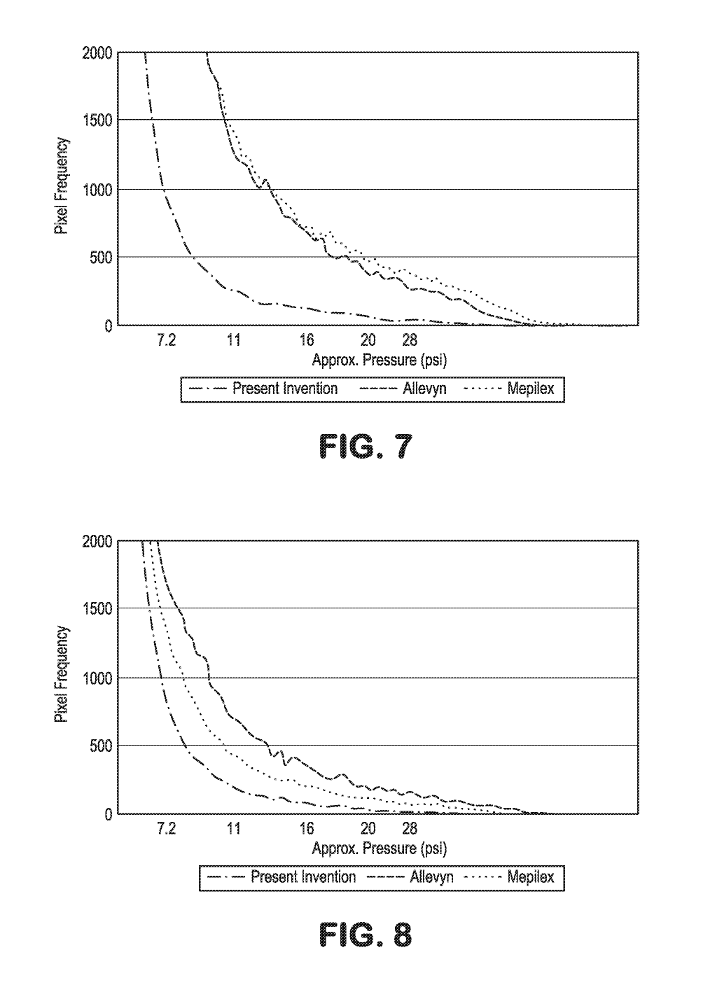

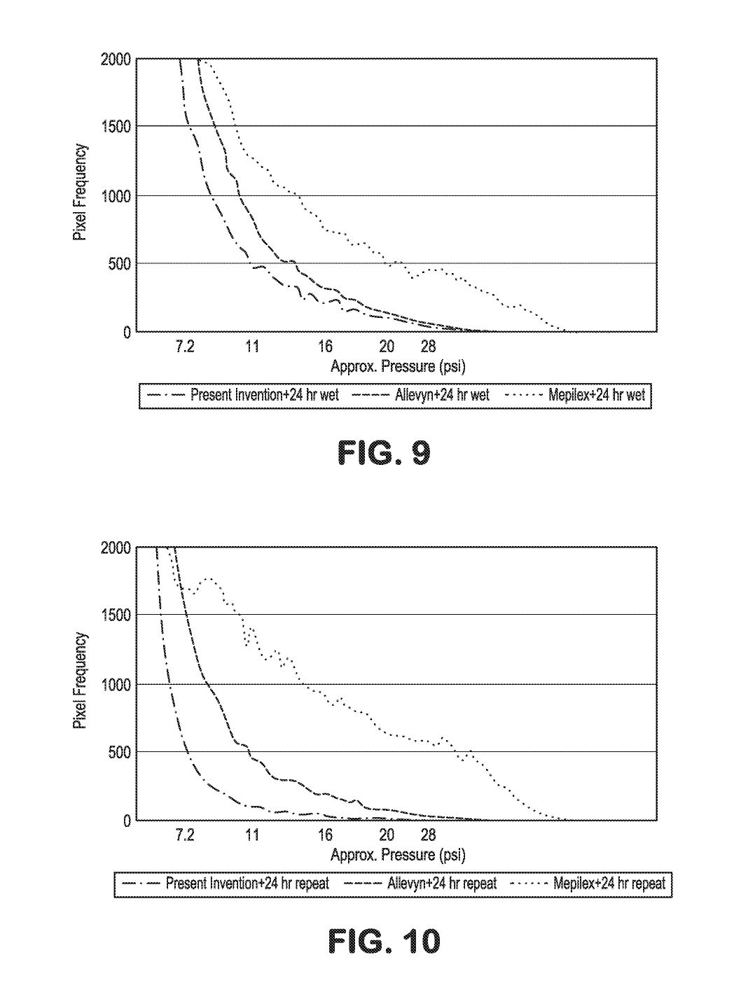

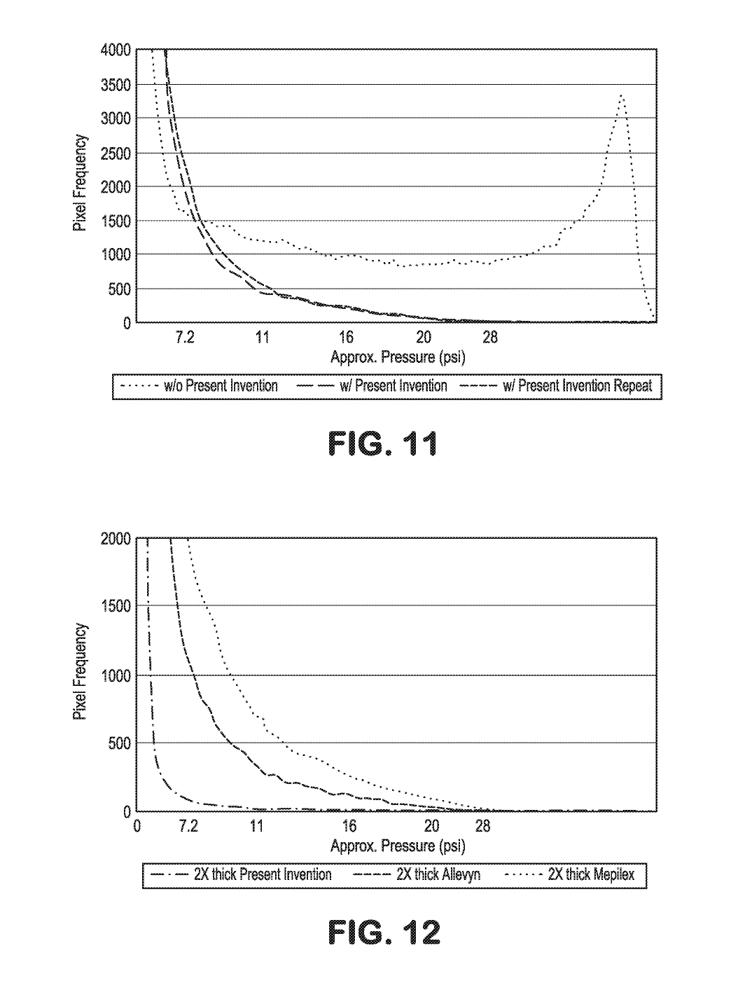

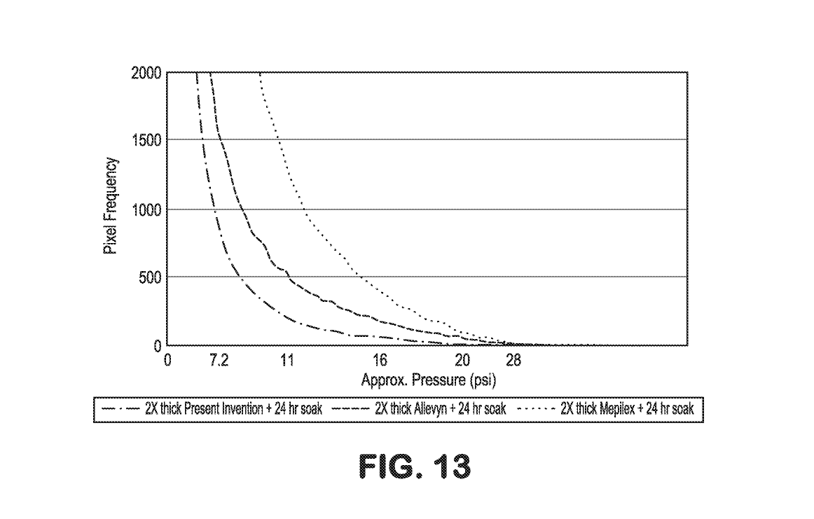

As further shown in FIG. 6, a system 600 including a 3.8 kg steel cylinder 610 (Diameter=1.75″, Height=12.0″) was used to apply force. A 0.04 kg solid aluminum cylinder 620 (Diameter=1.0″, Height=1.0″) was placed between the steel cylinder/weight 610 and the particular dressing being tested 630 to reduce the contact surface area to 5.1 cm², thereby increasing the theoretical pressure to 10.8 psi (74.5 kiloPascals). The secondary surface was a smooth concrete floor 660. For trials where the dressing 630 was wetted or moistened (FIGS. 9-10 and 13), the film 650 was covered with a thin polyethylene (PE) layer 640 to prevent water from contacting the film 650, and any effects created by this layer 640 were considered negligible. A dry foam dressing 630 was placed between the aluminum cylinder 620 and film 650 to absorb pressure. The steel cylinder/weight 610 was applied for 10 seconds and then removed. Ambient temperature and humidity was recorded using a calibrated hygrometer. The test was then performed with the remaining two dressings (see FIG. 7). This test was repeated using the same area on each dressing (see FIG. 8). This sequence was then repeated twice for dressings wetted for 24 hours in 0.9% saline (see FIG. 9 and FIG. 10). A control test was then performed to compare the pressure-distributing capabilities of the dressing/device of the present invention compared to no dressing being present (see FIG. 11). Then, the same testing summarized in FIGS. 7-10 (3 dry and 24-hour soak, 2 tests each) was then carried out using two-layer dressings, such that the dressings tested were twice as thick as the dressings tested in FIGS. 7-10 (see FIG. 12 and FIG. 13). In other words, the dressing of the present invention had a thickness of 0.52 inches (13.21 millimeters) and the Allevyn® and Mepilex® dressings each had a thickness of 0.44 inches (11.18 millimeters).

### Sample Analysis

The pressure mapping on the film 650 for each sample dressing was scanned and imported, including a scan of the color correlation chart, in order to minimize variation due to multiple scans. The TIFF file was opened in MS Paint and the regions of interest for each sample were selected and copied to an individual image, which was saved as a BMP file. Image J (NIH Java image processing program) was used to create a histogram by assigning pixels at different color intensities a bin value ranging from 0-255 (0=black, 255=white). The resulting output was copied to MS Excel, where the histograms were converted to scatterplots. The white background color at 255 was removed. In simple terms, a dressing having a lower pixel frequency corresponds with a dressing that is able to distribute pressure better/more evenly across the surface of the dressing. Peaks within the color correlation chart scan were assigned a 0.1-1.0 value relative to the color density of the film (0.1=least color, 1.0=most color). For example, the assigned value of 168 corresponded to a color density of 0.8. Values that fell between peaks were assigned a color density through linear extrapolation between the peaks of known value. The “Continuous Pressure Standard Charts”, provided by the FujiFilm Prescale® manufacturer, were used to correlate color density to pressure.

### Results

The duplicate tests for the dry dressings and 24 hour wetting time dressings were executed and image analysis was performed following the method above. The ambient temperature and humidity during testing was 24.9° C. and 49.5% RH, respectively. Frequency, or number of pixels grouped in a specific bin by the imaging software, was plotted against approximate pressure.

As shown in the chart in FIG. 7, the surface beneath the dry closed cell absorbent foam dressing of the present invention was subjected to 81% less average pressure than the surface beneath the dry Allevyn® dressing and 79% less average pressure than the surface beneath the dry Mepilex® dressing.

As shown in the chart in FIG. 8, in a repeat of the testing summarized by FIG. 7, the surface beneath the dry closed cell absorbent foam dressing of the present invention was subjected to 71% less average pressure than the surface beneath the dry Allevyn® dressing and 59% less average pressure than the surface beneath the dry Mepilex® dressing.

As shown in the chart in FIG. 9, the surface beneath the closed cell absorbent foam dressing of the present invention was subjected to 31% less average pressure than the surface beneath the Allevyn® dressing and 50% less average pressure than the surface beneath the Mepilex® dressing when the dressings were soaked for 24 hours in 0.9% saline.

As shown in the chart in FIG. 10, in a repeat of the testing summarized by FIG. 9, the surface beneath the closed cell absorbent foam dressing of the present invention was subjected to 78% less average pressure than the surface beneath the Allevyn® dressing and 84% less average pressure than the surface beneath the Mepilex® dressing when the dressings were soaked for 24 hours in 0.9% saline.

As shown in the chart in FIG. 11, the surface beneath the closed cell absorbent foam dressing of the present invention was subjected to significantly less pressure than the control sample surface, where the closed cell absorbent foam dressing of the present invention was not in place.

As shown in the chart in FIG. 12, the surface beneath the dry two-layer closed cell absorbent foam dressing of the present invention was subjected to 95% less average pressure than the surface beneath the dry two-layer Allevyn® dressing and 97% less average pressure than the surface beneath the dry two-layer Mepilex® dressing.

As shown in the chart in FIG. 13, the surface beneath the two-layer closed cell absorbent foam dressing of the present invention was subjected to 62% less average pressure than the surface beneath the two-layer Allevyn® dressing and 84% less average pressure than the surface beneath the two-layer Mepilex® dressing when the dressings were soaked for 24 hours in 0.9% saline.

In conclusion, the single-layer closed cell foam dressing tested was 0.04 inches thicker than both the Allevyn® and Mepilex® open cell foam dressings, which was beneficial in creating a slightly thicker barrier between the contacting surfaces. Further, despite the foam dressing having nearly four times less mass than the polyurethane dressings, the oxygen containing closed cells demonstrated better pressure absorption than the more dense polyurethane foams. On the surface, this observation may be counterintuitive, however, it is thought that the trapped oxygen within the closed cells of the dressing of the present invention provides greater resiliency, whereas the polyurethane foams collapsed once the weight was applied.

### Example 3: Pressure Distribution Testing

### Background

The experimental setup described above in Example 2 and illustrated in FIG. 6 was utilized to test additional dressings. Dressings in the “Simulated Use” condition were wetted following the procedure set forth in the “Free Swell Absorptive Capacity” section of BS EN 13726-1:2002 “Test methods for primary wound dressings.” This method used 40× standardized saline with a 30 minute exposure time and an exposure temperature of 37° C. The various dressings tested are listed below in Table 7.

TABLE 7
Typical weight and thickness of dressings used during testing.

| | Thickness | | |
|---|---|---|---|
| Dressing | Condition | Dry Weight (g) | Dry Thickness (mm) |
| Allevyn ® | Single Layer | 6.5 | 4.8 |
| Allevyn ® | Double Layer | 12.8 | 10.1 |
| Mepilex ® | Single Layer | 6.3 | 5.0 |
| Mepilex ® | Double Layer | 12.3 | 10.0 |
| Present Invention | Single Layer | 1.4 | 5.3 |
| Present Invention | Double Layer | 2.9 | 10.7 |

### Sample Analysis

Films were scanned on a HP LaserJet M4555 with the following specifications: High Quality, 600 dpi, color, and saved as TIFF file. The TIFF file was opened in Paint and the regions of interest (1001 pixels×1001 pixels) for each dressing sample were selected and copied to an individual image, saved as BMP. Image J (NIH Java image processing program) was used to create a histogram by assigning pixels at different color intensities a bin value of 0-255 (0=black, 255=white). The resulting output was copied to Excel for further analysis. A blank piece of film was analyzed following the same method to determine the effect of the white background. All pixels of this control film registered a value of 255. Pixels at the 255 value were discarded from the sample analysis to remove the white background.

Peaks within the color correlation chart scan were assigned a 0.1-1.0 value relative to the color density of the film (0.1=least color, 1.0=most color). For example, the assigned value of 168 corresponded to a color density of 0.8. Values that fell between peaks were assigned a color density through linear interpolation between the peaks of known value. The “Continuous Pressure Standard Charts,” provided by the FujiFilm Prescale® manufacturer, were used to correlate color density to pressure. Note that the x-axis (Pressure) for the analysis is not linear, as the color charts provided by the manufacturer were not linear. Interpolation between the 6 pressures listed was not possible.

### Results

Dry Testing

Dry dressing samples were tested in triplicate for each thickness condition listed in Table 7 above. The image analysis of the pressure films, described above, produced line graphs showing number of pixels vs. pressure (psi) to represent the color distribution on the film.

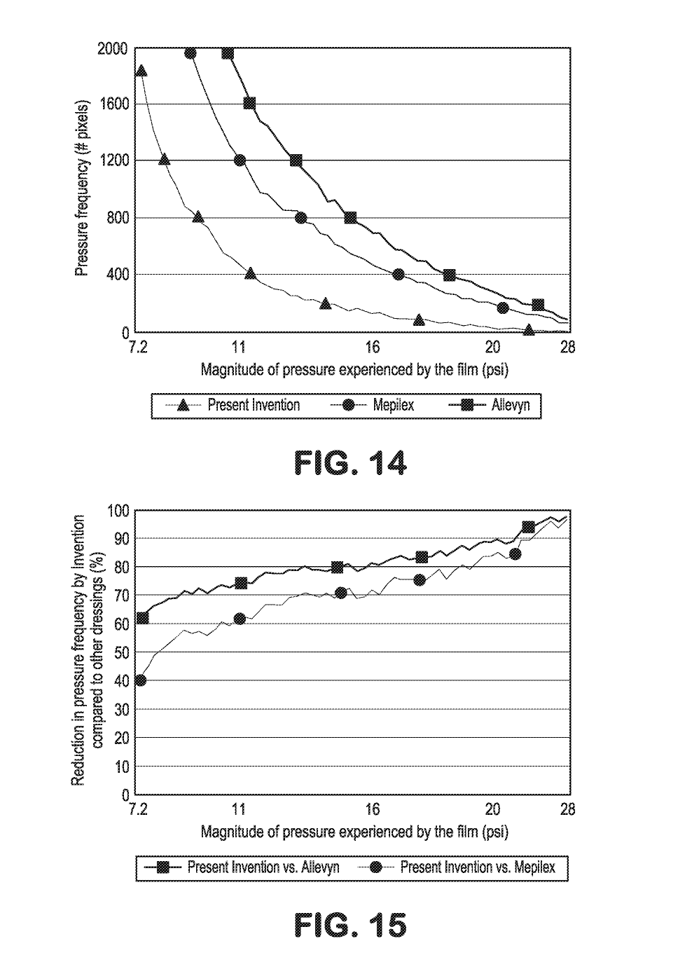

FIG. 14 shows a comparison for the present invention, Allevyn®, and Mepilex® foam wound dressings at the dry, single-layer thickness condition (n=3). Each curve represents an average of 3 trials. Curves with lower pressure frequency (number of pixels on the film) represent better pressure redistributing properties, since less of the applied pressure is translated to the film. As shown, the dressing of the present invention displays the greatest ability to redistribute pressure under this condition, as represented by the curve with triangles. In summary, the dressing of the present invention (see line with triangles) demonstrated the best pressure redistributing ability as compared to the Allevyn® dressing (see line with squares) and the Mepilex® dressing (see line with circles).

FIG. 15 illustrates a comparison between the a dry, single-layer thickness dressing of the present invention and the dry, single-layer thickness Allevyn® dressing and the dry, single-layer thickness dressing of the present invention and the dry, single-layer thickness Mepilex® dressing. Each curve represents the percent reduction in pressure frequency (number of pixels on the film) between the dressing of the present invention and the other dressings. At the dry, single-layer thickness condition, the dressing of the present invention reduces pressure frequency by 61%-98% compared to Allevyn® (see line with squares) and 40%-97% compared to Mepilex® (see line with circles). The most significant improvement occurred at the high end of pressure experienced by the film. For example, the present invention is shown to decrease pressure frequency by approximately 80% compared to Allevyn® at 16 psi (110 kiloPascals).

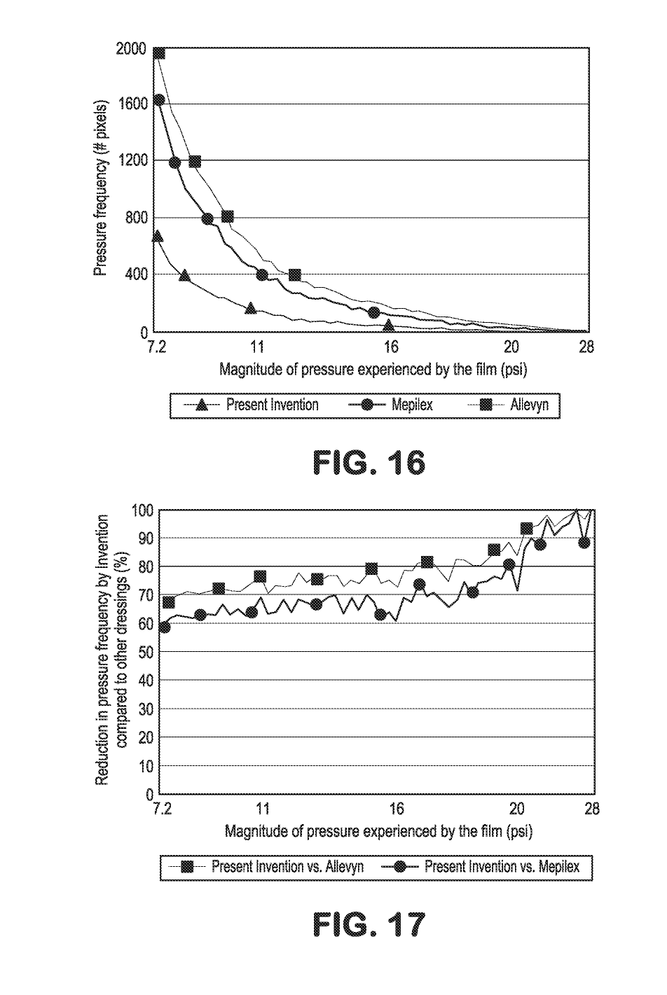

FIG. 16 shows a comparison for the present invention, Allevyn®, and Mepilex® foam wound dressings at the dry, double-layer thickness condition (n=3). Each curve represents an average of 3 trials. Curves with lower pressure frequency (number of pixels on the film) represent better pressure redistributing properties, since less of the applied pressure is translated to the film. As shown, the dressing of the present invention displays the greatest ability to redistribute pressure under this condition, as represented by the curve with triangles. In summary, the dressing of the present invention (see line with triangles) demonstrated the best pressure redistributing ability as compared to the Allevyn® dressing (see line with squares) and the Mepilex® dressing (see line with circles).

FIG. 17 illustrates a comparison between the a dry, double-layer thickness dressing of the present invention and the dry, double-layer thickness Allevyn® dressing and the dry, double-layer thickness dressing of the present invention and the dry, single-layer thickness Mepilex® dressing. Each curve represents the percent reduction in pressure frequency (number of pixels on the film) between the dressing of the present invention and the other dressings. At the dry, double-layer thickness condition, the dressing of the present invention reduces pressure frequency by 66%-100% compared to Allevyn® (see line with squares) and 59%-100% compared to Mepilex® (see line with circles). The most significant improvement occurred at the high end of pressure experienced by the film. For example, the present invention is shown to decrease pressure frequency by approximately 70% compared to Mepilex® at 11 psi (75.8 kiloPascals). Please note that FIG. 17 displays multiple peaks and valleys, which are believed to be due to the low pixel count at the high end of the pressure scale.

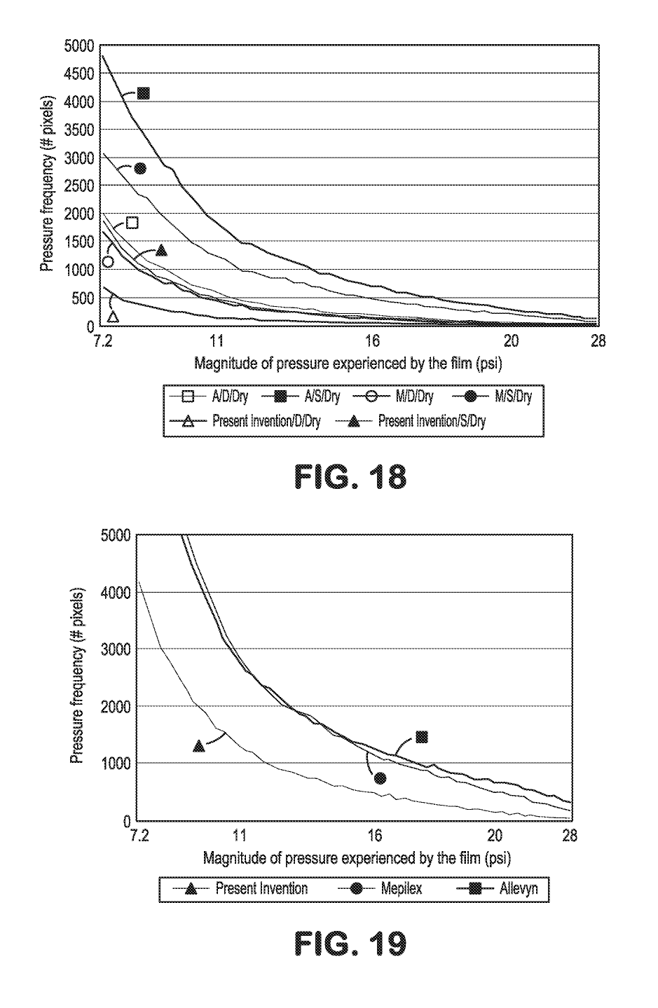

FIG. 18 is a summary of the output of image analysis plotted for the present invention, Mepilex®, and Allevyn® dressings at the dry, single-layer thickness and the dry, double-layer thickness conditions. Each curve represents an average of 3 trials. Curves with lower pressure frequency (number of pixels on the film) represent better pressure redistributing properties, since less of the applied pressure is translated to the film. For all dry dressings tested, the double-layer thickness condition performed better than the single-layer thickness condition to redistribute pressure. This result is consistent with previous studies and the notion that a thicker dressing provides a better barrier layer to pressure. Surprisingly, the single-layer thickness dressing of the present invention performed almost identically to the Mepilex® double-layer thickness and Allevyn® double-thick dressings. At 5.3 mm thick, the dressing of the present invention was nearly half the thickness of the double-layer thickness Mepilex® (10.0 mm) and the double-thick Allevyn® (10.1 mm). This observation demonstrates the efficiency by which the dressing of the present invention is able to redistribute pressure.

Moist Testing

Wetting of the dressing samples followed the “Free Swell Absorptive Capacity” section of BS EN 13726-1:2002 “Test methods for primary wound dressings.” The test method exposed a 5×5 cm dressing sample to 40× saline for a time period of 30 minutes and at a temperature of 37° C. This condition represented a clinically relevant amount of saline absorption for the dressing in order to test how they redistribute pressure. FIGS. 19-23 show the testing results of all moist dressing samples.

FIG. 19 shows a comparison for the present invention, Allevyn®, and Mepilex® foam wound dressings at the moist, single-layer thickness condition (n=3). Each curve represents an average of 3 trials. Curves with lower pressure frequency (number of pixels on the film) represent better pressure redistributing properties, since less of the applied pressure is translated to the film. As shown, the dressing of the present invention displays the greatest ability to redistribute pressure under this condition, as represented by the curve with triangles. In summary, the dressing of the present invention (see line with triangles) demonstrated the best pressure redistributing ability as compared to the Allevyn® dressing (see line with squares) and the Mepilex® dressing (see line with circles).

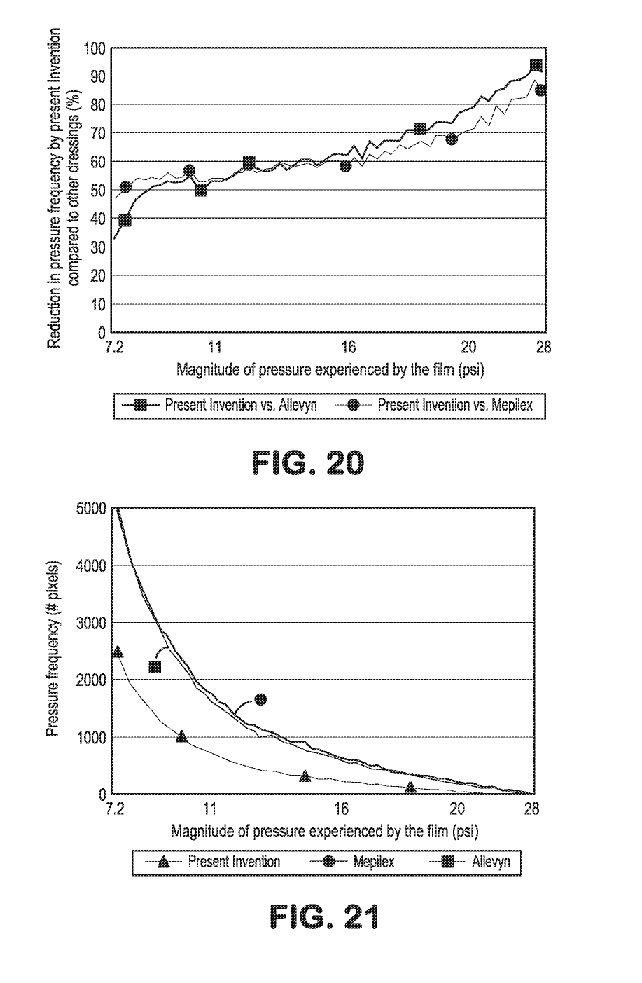

FIG. 20 illustrates a comparison between the a moist, single-layer thickness dressing of the present invention and the moist, single-layer thickness Allevyn® dressing and the moist, single-layer thickness dressing of the present invention and the moist, single-layer thickness Mepilex® dressing. Each curve represents the percent reduction in pressure frequency (number of pixels on the film) between the dressing of the present invention and the other dressings. At the moist, single-layer thickness condition, the dressing of the present invention reduces pressure frequency by 33%-92% compared to Allevyn® (see line with squares) and 47%-84% compared to Mepilex® (see line with circles). The most significant improvement occurred at the high end of pressure experienced by the film. For example, the present invention is shown to decrease pressure frequency by approximately 65% compared to Allevyn® at 16 psi (110 kiloPascals).

FIG. 21 shows a comparison for the present invention, Allevyn®, and Mepilex® foam wound dressings at the moist, double-layer thickness condition (n=3). Each curve represents an average of 3 trials. Curves with lower pressure frequency (number of pixels on the film) represent better pressure redistributing properties, since less of the applied pressure is translated to the film. As shown, the dressing of the present invention displays the greatest ability to redistribute pressure under this condition, as represented by the curve with triangles. In summary, the dressing of the present invention (see line with triangles) demonstrated the best pressure redistributing ability as compared to the Allevyn® dressing (see line with squares) and the Mepilex® dressing (see line with circles).

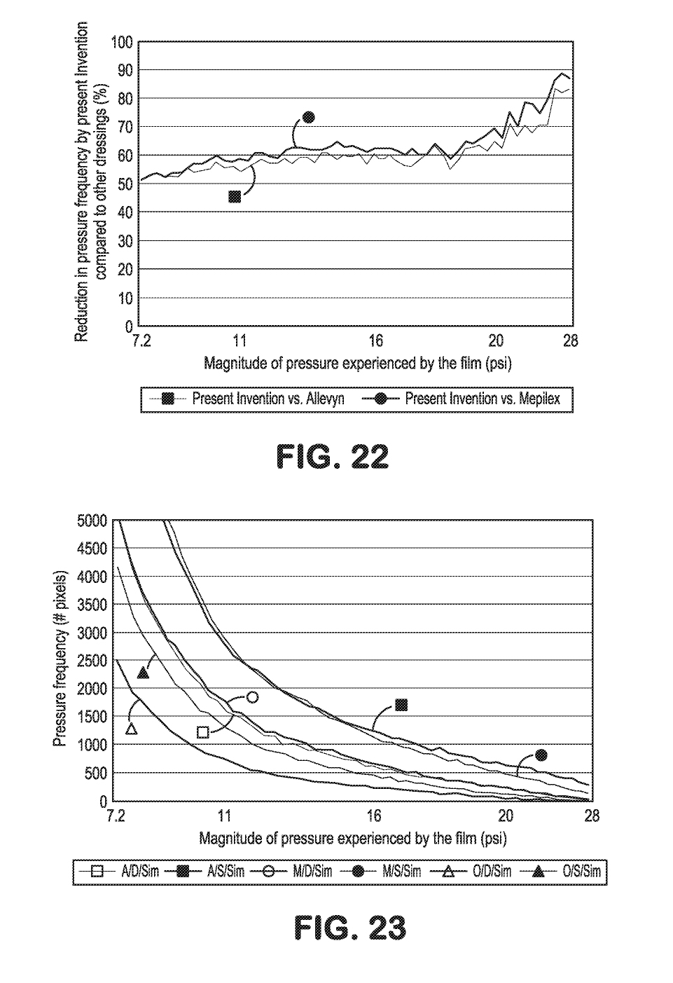

FIG. 22 illustrates a comparison between the a moist, double-layer thickness dressing of the present invention and the moist, double-layer thickness Allevyn® dressing and the moist, double-layer thickness dressing of the present invention and the moist, double-layer thickness Mepilex® dressing. Each curve represents the percent reduction in pressure frequency (number of pixels on the film) between the dressing of the present invention and the other dressings. At the moist, double-layer thickness condition, the dressing of the present invention reduces pressure frequency by 52%-83% compared to Allevyn® (see line with squares) and 51%-87% compared to Mepilex® (see line with circles). The most significant improvement occurred at the high end of pressure experienced by the film. For example, the present invention is shown to decrease pressure frequency by approximately 70% compared to Mepilex® at 11 psi (75.8 kiloPascals).

FIG. 23 is a summary of the output of image analysis plotted for the present invention, Mepilex®, and Allevyn® dressings at the moist, single-layer thickness and the moist, double-layer thickness conditions. Each curve represents an average of 3 trials. Curves with lower pressure frequency (number of pixels on the film) represent better pressure redistributing properties, since less of the applied pressure is translated to the film. For all moist dressings tested, the double-layer thickness condition performed better than the single-layer thickness condition to redistribute pressure. This result is consistent with previous studies and the notion that a thicker dressing provides a better barrier layer to pressure. For all moist dressings tested, the double-thick condition performed better than the single-thick condition to redistribute pressure. This result is consistent with previous studies and the notion that a thicker dressing provides a better barrier layer to pressure, even under moist conditions. Similar to the dry testing, the single-layer thickness dressing of the present invention meets or exceeds the pressure redistributing capabilities of the Mepilex® double-layer thickness and Allevyn® double-layer thickness dressings. Interestingly, Mepilex® and Allevyn® foams performed almost identically whether the dressing had a single-layer thickness or a double-layer thickness.

### CONCLUSIONS

This Example investigated the ability of the dressing of the present invention, the Allevyn® dressing, and the Mepilex® dressing to redistribute the pressure from an applied load. The dressing of the present invention demonstrated the best pressure redistributing ability at all conditions (dry, wet, single-layer thickness, and double-layer thickness) and the results for all conditions are summarized in Table 8.

Table 8: Percentage of pressure reduction by Invention compared to Allevyn and Mepilex for all conditions tested.

TABLE 8
Percentage of pressure reduction by Invention compared
to Allevyn and Mepilex for all conditions tested.

| Present | | | | |
|---|---|---|---|---|
| Invention | Dry/ | Dry/ | Moist/ | Moist/ |
| Dressing | Single- | Double- | Single- | Double- |
| Compared | Layer | Layer | Layer | Layer |
| to: | Thickness | Thickness | Thickness | Thickness |
| Allevyn ® | 61-98% | 66-100% | 33-92% | 52-83% |
| Mepilex ® | 40-97% | 59-100% | 47-84% | 51-87% |

In summary, the dressing of the present invention exhibits unique pressure redistributing qualities due to the specifically controlled oxygen-filled closed cell structure of the foam. This composition gives the foam a more rigid, supportive structure compared to the open cell foams which collapse under pressure. These results suggest that dressing of the present invention has superior pressure redistributing qualities and could improve the prevention of pressure ulcers in a clinical setting.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A pressure-distributing device comprising a biocompatible polymeric matrix, wherein the matrix is hydrophilic and comprises a polymer and closed cells, wherein the device distributes pressure away from an area of intact skin or a wound, wherein the device is configured to directly contact the area of intact skin or the wound.

2. The device of claim 1, wherein the intact skin or the wound is subjected to a resulting pressure level ranging from about 0 pounds per square inch (0 kiloPascals) to about 5 pounds per square inch (34.5 kiloPascals).

3. The device of claim 1, wherein a load having a pressure level ranging from about 1 pound per square inch (6.9 kiloPascals) to about 50 pounds per square inch (345 kiloPascals) is applied to the pressure-distributing device.

4. The device of claim 1, wherein the device has a thickness ranging from about 0.1 inches (2.54 millimeters) to about 1 inch (25.4 millimeters).

5. The device of claim 1, wherein the matrix absorbs moisture from the area of intact skin or the wound.

6. The device of claim 1, wherein the matrix supplies moisture to the area of intact skin or the wound.

7. The device of claim 1, wherein the polymer is at least partially cross-linked.

8. The device of claim 1, wherein the polymer is an absorbent, elastomeric, and biocompatible polymer or copolymer.

9. The device of claim 8, wherein the absorbent, elastomeric, and biocompatible polymer or copolymer comprises polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof.

10. The device of claim 8, wherein the device further comprises a polysaccharide, wherein the polysaccharide comprises dextrin, guar gum, lucerne, fenugreek, xanthan gum, honey locust bean gum, white clover bean gum, carob locust bean gum, carrageenan, sodium alginate, chitin, chitosan, cellulose, or a combination thereof.

11. (canceled)

12. The device of claim 1, wherein the device contains a gas, wherein the gas is contained in the closed cells, wherein the gas comprises oxygen, nitrogen, nitric oxide, carbon dioxide, air, helium, or a combination thereof.

13. The device of claim 12, wherein a dissolved form of the gas is contained in or between the closed cells.

14. (canceled)

15. The device of claim 1, wherein the device further comprises an active agent, wherein the active agent comprises an antimicrobial agent, an antifungal agent, an antifouling agent, an analgesic, a deodorizing agent, nanoparticles, nutrients, growth factors, biologics, or a combination thereof.

16. (canceled)

17. The device of any of claim 1, wherein the device further comprises a superabsorbent material.

18. The device of claim 1, wherein the device is two-dimensional.

19. The device of claim 1, wherein the device is three-dimensional and seamless, wherein the device conforms to a part of the body having non-planar geometry.

20. The device of claim 18, wherein the part of the body is a finger, toe, hand, foot, knee, elbow, heel, amputee stump, or sacral area.

21. The device of claim 1, wherein the device comprises one or more layers of the biocompatible polymeric matrix.

22. The device of any of claim 1, wherein the device further comprises open cells.

23. A method for preventing or treating pressure sores around an area of intact skin or a wound, the method comprising:
directly applying a pressure-distributing device to the intact skin or the wound, wherein the pressure-distributing device comprises a biocompatible polymeric matrix, wherein the matrix is hydrophilic and comprises a polymer and closed cells, wherein the device distributes pressure away from the area of intact skin or the wound.

24. The method of claim 23, wherein the intact skin or the wound is subjected to a resulting pressure level ranging from about 0 pounds per square inch (0 kiloPascals) to about 5 pounds per square inch (34.5 kiloPascals).

25. The method of claim 23, wherein a load having a pressure level ranging from about 1 pound per square inch (6.9 kiloPascals) to about 50 pounds per square inch (345 kiloPascals) is applied to the pressure-distributing device.

26-44. (canceled)

45. A system for preventing or treating pressure sores around an area of intact skin or a wound, the system comprising a biocompatible polymeric matrix, wherein the matrix is hydrophilic and comprises a polymer and closed cells, wherein the system distributes pressure away from the area of intact skin or the wound, manages moisture surrounding the area of intact skin or the wound, and delivers a therapeutic agent to tissue at or beneath the area of intact skin or the wound.

46. The method of claim 45, wherein the intact skin or the wound is subjected to a resulting pressure level ranging from about 0 pounds per square inch (0 kiloPascals) to about 5 pounds per square inch (34.5 kiloPascals).

47. The system of claim 45, wherein a load having a pressure level ranging from about 1 pound per square inch (6.9 kiloPascals) to about 50 pounds per square inch (345 kiloPascals) is applied to the system.

48-66. (canceled)
